(19) United States
(12) Patent Application Publication
(10) Pub. No.: US 2023/0349906 A1
(43) Pub. Date: Nov. 2, 2023

(54) KINASES AS BIOMARKERS FOR NEURODEGENERATIVE CONDITIONS

(71) Applicant: Chase Therapeutics Corporation, Washington, DC (US)

(72) Inventors: Thomas N. Chase, Washington, DC (US); Kathleen Clarence-Smith, Washington, DC (US)

(21) Appl. No.: 17/789,423

(22) PCT Filed: Dec. 29, 2020
(86) PCT No.: PCT/US20/67368

§ 371 Date: Jun. 27, 2022

Related U.S. Application Data
(60) Provisional application No. 62/956,029, filed on Dec. 31, 2019.

(51) Int. Cl. G01N 33/573; G01N 33/68; A61K 31/5377; A61K 31/426; A61P 25/28; A61K 31/366; G16H 10/40; G16H 50/70; G16H 70/60

(52) U.S. Cl. CPC G01N 33/573; G01N 33/6896; A61K 31/5377; A61K 31/426; A61P 25/28; A61K 31/366; G16H 10/40; G16H 50/70; G16H 70/60; G01N 2333/912; G01N 2800/2814; G01N 2800/2835

(57) ABSTRACT

An assay for using signaling kinases alone or in combination with oligomeric forms of neurodegenerative proteins can include: a) providing a biological sample, e.g., a blood sample, from a subject; b) enriching for neuronally (e.g., central nervous system (“CNS”)) derived microparticles, e.g., exosomes, from the blood sample; c) removing proteins from the surface of the isolated exosomes to produce scrubbed exosomes; d) determining, in the isolated internal contents, set of biomarkers including: (1) at least one signaling kinase and, optionally, at least one oligomeric form of a neurodegenerative protein; or (2) a plurality of different signaling kinases.

### REFERENCE TO RELATED APPLICATIONS

This application claims the benefit of the priority date of U.S. Provisional application 62/956,029, filed Dec. 31, 2019, the contents of which are incorporated herein in their entirety.

### BACKGROUND

Neurodegenerative diseases are characterized by degenerative changes in the brain including loss of function and death of neurons. Neurodegenerative diseases include, without limitation, Parkinson's disease, Alzheimer's disease, Huntington's disease, amyotrophic lateral sclerosis and Lewy Body dementia.

Various signaling kinases have been implicated in neurodegenerative diseases. See, for example, Mehdi, S. J. et al., “Protein Kinases and Parkinson's Disease,” Int J Mol Sci. 2016 September; 17(9): 1585 (doi: 10.3390/ijms17091585); Martin, L. et al., “Tau protein kinases: Involvement in Alzheimer's disease,” Ageing Research Reviews, Volume 12, Issue 1, January 2013, Pages 289-309 (doi.org/10.1016/j.arr.2012.06.003); and Bowles, K. R. et al., “Kinase Signaling in Huntington's Disease,” Journal of Huntington's Disease 3 (2014) 9-123 (DOI 10.3233/JHD-140106).

Many neurodegenerative diseases are characterized by the aberrant accumulation of oligomeric forms of proteins. It is believed that these oligomeric forms contribute to neuronal degeneration and death. In particular, Parkinson's Disease is characterized by accumulation of oligomeric forms of alpha synuclein. It has further been found that alpha synuclein can aggregate to form co-polymers with other proteins, such as tau and amyloid beta.

### SUMMARY OF THE DISCLOSURE

Referring to FIG. 1, assays for kinases include the following operations: A body fluid sample, such as a blood or saliva sample from a subject is obtained (100). The blood sample may be treated to provide a blood fraction, e.g., a plasma sample. The blood sample is enriched for neuronally-derived microparticles, e.g., exosomes (e.g., neuronally derived exosomes are isolated from the blood sample) (110). This can be a two-step operation that involves, first, isolating total exosomes (111) and, second, enriching neuronally derived exosomes from the total exosomes (112). Isolated exosomes are enriched for their internal contents (120). This can involve scrubbing to remove proteins attached to their surfaces (121). The internally enriched contents of the exosomes are released for analysis (122). Analysis involves measuring in the sample biomarkers selected from either (1) amounts of at least one signaling kinase and, optionally, at least one oligomeric form of a neurodegenerative protein (e.g., oligomeric alpha synuclein), or (2) amounts of (e.g., activity of) each of a plurality of different signaling kinases. Optionally, amounts of one or more forms of neurodegenerative proteins, e.g., monomeric α synuclein and/or oligomeric a synuclein and tau or amyloid beta, also can be measured. Measurement of kinases, in combination with oligomeric forms, can reduce false positive classifications.

Measures of these biomarkers, can be used in diagnostic testing to determine presence or absence of a particular neurodegenerative condition (e.g., a syncucleinopathic condition) or of its cumulative severity or current rate of progression, or to determine efficacy of a drug to alter amounts or relative amounts of one or more biomarker proteins described herein toward normal amounts.

Disclosed herein are, among other things, biomarker profiles for neurodegenerative conditions, such as syncucleinopathic conditions, amyloidopathic conditions, tauopathies and Huntington's disease, and the neurodegeneration associated therewith. In certain embodiments, the biomarker profiles comprise measures of a set of biomarkers that include at least one signaling kinase and that can be selected from (1) at least one signaling kinase and, optionally, at least one oligomeric form of a neurodegenerative protein, or (2) each of one or a plurality of different signaling kinases. Biomarker profiles can comprise measures of one or more oligomeric forms of neurodegenerative proteins, such as alpha-synuclein, amyloid beta, tau or huntingtin.

Signaling kinases measured can be one or a plurality of kinases. They can be selected from the same signaling pathway, such as the mTOR pathway, or from different signaling pathways.

Oligomeric forms of neurodegenerative proteins measured can be a collection of forms, such as total oligomeric alpha synuclein, or individual oligomeric forms, such as a tetramer of alpha synuclein, or a plurality of forms, such as alpha synuclein dimers, trimers and tetramers. Monomeric forms of the neurodegenerative protein also can be measured. So, for example, the biomarker profile can comprise measures of each of one or a plurality of neurodegenerative protein forms selected from: (I) at least one oligomeric form; (II) a plurality (e.g., pattern) of oligomeric forms; (Ill) at least one oligomeric form and at least one monomeric form; (IV) a plurality of oligomeric forms and at least one monomeric form; (V) at least one oligomeric form and a plurality of monomeric forms; and (VI) a plurality of oligomeric forms and a plurality of monomeric forms.

Further disclosed herein are methods of developing pharmaceuticals for treatment of neurodegenerative conditions, such as syncucleinopathic conditions, amyloidopathic conditions, tauopathic conditions, and Huntington's disease. The methods involve using a biomarker profile to determine the effect of a candidate pharmaceutical on the condition. The biomarker profile includes measures of a biomarker set including biomarkers selected from (1) at least one signaling kinase and, optionally, at least one oligomeric form of a neurodegenerative protein, or (2) each of one or a plurality of different signaling kinases. Biomarker proteins can be quantified from, e.g., neuronally derived microparticles, e.g., exosomes, from the blood of a subject.

In certain embodiments, the protein species are measured from neuronally derived extracellular vesicles, hereinafter termed exosomes, isolated, e.g., from blood, saliva, or urine. The species examined can derive from an internal compartment of the exosome, e.g., from exosomes from which surface proteins have been removed. The biomarker profiles, measured in this way, represent a relatively simple and non-invasive means for measurement.

As such, methods of this disclosure for measuring a biomarker profile for a neurodegenerative condition are useful in drug development for testing neuroprotective efficacy of a drug candidate, sometimes referred to herein as a putative neuroprotective agent. For example, the methods described herein can be used to further understand the downstream effects of kinase activity, and to accelerate the development of effective therapeutic strategies. Such methods also are useful for identifying subjects for enrollment in clinical trials and for determining a diagnosis, prognosis, progression or risk of developing a syncucleinopathic condition. Further provided herein are novel methods of treating a subject determined, by the methods of this disclosure, to have or to be at risk of developing neurodegeneration associated with syncucleinopathic conditions, in particular, a neuroprotective treatment.

In one embodiment provided herein is a method comprising: a) enriching each biological sample in a collection of biological samples for neuronally derived microparticles, e.g., exosomes, wherein: (i) the collection of biological samples is from subjects in a cohort of subjects, wherein the cohort comprises subjects including: (1) a plurality of subjects diagnosed with a neurodegenerative condition at each of a plurality of different disease stages, wherein each of the diagnosed subjects has received a putative neuroprotective agent, and/or (2) a plurality of healthy control subjects, wherein the biological samples were collected before and again at one or more times during and, optionally, after administration of the putative neuroprotective agent; b) isolating protein contents from an internal compartment of the microparticles, e.g., exosomes, to produce a biomarker sample; c) measuring, in the biomarker sample, a set of biomarkers to create a dataset, wherein the set of biomarkers includes: (i) at least one signaling kinase and, optionally, at least one oligomeric form of a neurodegenerative protein; or (ii) a plurality of different signaling kinases; and d) performing statistical analysis on the dataset to compare differences in the biomarker sets: (i) in individual subjects over time to determine a diagnostic algorithm that predicts rates of disease progression or degree of response to the putative neuroprotective agent; or (ii) between different subjects to determine a diagnostic algorithm that (1) makes a pathogenic diagnosis, (2) separates clinically similar but etiologically different neurodegenerative disorder subgroups, or (3) predicts whether or the degree to which a subject is likely to respond to the putative neuroprotective agent. In one embodiment the method further comprises, before enriching: I) providing a cohort of subjects, wherein the cohort comprises subjects including: (i) a plurality of subjects diagnosed with a neurodegenerative condition at each of a plurality of different disease stages, and/or (ii) a plurality of healthy control subjects; II) administering to each of the diagnosed subjects a putative neuroprotective agent; Ill) before and again at one or more times during and, optionally, after administration of the putative neuroprotective agent, collecting a biological sample from each of the subjects in the cohort. In another embodiment at least one of the signaling kinases is a kinase of the PI3K-Akt-mTOR signaling pathway. In another embodiment wherein at least one of the signaling kinases is selected from mitogen-activated protein kinase (MAPK or MEK), extracellular signal-regulated kinases (ERK), glycogen synthase kinase 3 beta (GSK3B), AKT kinase and beclin. In another embodiment wherein the neurodegenerative protein selected from alpha synuclein, amyloid beta, tau, or huntingtin. In another embodiment the oligomeric form of the neurodegenerative protein is a collection of oligomeric forms, e.g., oligomers of alpha synuclein, e.g., alpha synuclein 2-50, e.g., alpha synuclein 4-30, e.g., alpha synuclein 4-20. In another embodiment the method further comprises: e) validating one or more of the diagnostic algorithms against standard clinical measures. In another embodiment wherein the statistical analysis comprises: correlational, Pearson correlation, Spearman correlation, chi-square, comparison of means (e.g., paired T-test, independent T-test, ANOVA) regression analysis (e.g., simple regression, multiple regression, linear regression, non-linear regression, logistic regression, polynomial regression. stepwise regression, ridge regression, lasso regression, elasticnet regression) or non-parametric analysis (e.g., Wilcoxon rank-sum test, Wilcoxon sign-rank test, sign test). In another embodiment the statistical analysis is executed by computer. In another embodiment wherein the statistical analysis comprises machine learning. In another embodiment the subjects are humans. In another embodiment the neurodegenerative condition is a syncucleinopathic disorder. In another embodiment the syncucleinopathic disorder is Parkinson's disease. In another embodiment the syncucleinopathic disorder is Lewy body dementia. In another embodiment the standard clinical measures are selected from UPDRS scores, CGI scores and radiologic findings. In another embodiment the neurodegenerative condition is an amyloidopathy, a tauopathy or Huntington's disease. In another embodiment the biological sample comprises a venous blood sample. In another embodiment the different disease stages comprise one or more of suspected, early, middle, and clinically advanced. In another embodiment the times during or after administration are selected from 1, 2, 3 or more months after treatment. In another embodiment enriching comprises using one or more brain-specific protein markers. In another embodiment at least one of the brain-specific markers comprises K1cam. In another embodiment isolating comprises washing the exosomes in each enriched sample to remove surface membrane-bound proteins. In another embodiment the exosomes are washed with PBS. In another embodiment the forms of the neurodegenerative protein are measured by gel electrophoresis, Western blot or fluorescence techniques.

In another aspect provided herein is a method comprising: a) enriching a biological sample from a subject for neuronally derived microparticles, e.g., exosomes; b) isolating protein contents from an internal compartment of the microparticles, e.g., exosomes, to produce a biomarker sample; c) measuring, in the biomarker sample, a set of biomarkers to create a dataset, wherein the set of biomarkers includes: (1) at least one signaling kinase and, optionally, at least one oligomeric form of a neurodegenerative protein; or (2) a plurality of different signaling kinases; and d) using the dataset to perform one of the following: (1) make a pathogenic diagnosis, (2) classify the subject into one of a plurality of clinically similar but etiologically different neurodegenerative disorder subgroups, or (3) predict whether or the degree to which the subject is likely to respond to the putative neuroprotective agent. In another embodiment using comprises executing a diagnostic algorithm as described herein, on the dataset. In another embodiment at least one of the signaling kinases is a kinase of the PI3K-Akt-mTOR signaling pathway. In another embodiment wherein at least one of the signaling kinases is selected from mitogen-activated protein kinase (MAPK or MEK), extracellular signal-regulated kinases (ERK), glycogen synthase kinase 3 beta (GSK3B), AKT kinase and beclin. In another embodiment the neurodegenerative protein selected from alpha synuclein, amyloid beta, tau, or huntingtin. In another embodiment the oligomeric form of the neurodegenerative protein is a collection of oligomeric forms, e.g., oligomers of alpha synuclein, e.g., alpha synuclein 2-50, e.g., alpha synuclein 4-30, e.g., alpha synuclein 4-20. In another embodiment isolating neuronally derived exosomes comprises: (i) ultra-centrifugation; (ii) density gradient centrifugation; or (iii) size exclusion chromatography. In another embodiment isolating neuronally derived exosomes comprises capturing the neuronally derived exosomes using a binding moiety that binds to brain-specific protein. In another embodiment the brain-specific protein is L1CAM. In another embodiment removing proteins from the surface of the isolated exosomes comprises washing the isolated exosomes with an aqueous solution (e.g., phosphate buffered saline (“PBS”)). In another embodiment determining amounts of a neurodegenerative protein comprises: i) separating species of oligomeric α-synuclein into a plurality of fractions; ii) measuring each of one or a plurality of the separated oligomeric α-synuclein species and, optionally, one or a plurality of species selected from: monomeric α-synuclein, tau-synuclein co-polymers, amyloid beta-synuclein co-polymers and tau-amyloid beta-synuclein co-polymers. In another embodiment separating species into a plurality of fractions comprises separating by electrophoresis. In another embodiment separating species into a plurality of fractions comprises separating by chromatography. In another embodiment determining among the separated species, at least one oligomeric form of α-synuclein selected from forms having between 2 and about 100 monomeric units, between 4 and 16 monomeric units and no more than about 30 monomeric units. In another embodiment determining among the separated species, a quantitative measure of monomeric α-synuclein. In another embodiment measuring among the separated species, a plurality of different oligomeric α-synuclein species. In another embodiment measuring among the separated species a co-polymer comprising α-synuclein and tau. In another embodiment determining among the separated species, a quantitative measure of a co-polymer comprising α-synuclein and amyloid beta. In another embodiment measuring the separated species comprises detecting one or a plurality of separated species by immunoassay. In another embodiment the immunoassay comprises immunoblotting. In another embodiment the immunoassay comprises Western blot. In another embodiment the immunoassay uses an antibody coupled to a direct label. In another embodiment the immunoassay uses an antibody coupled to an indirect label. In another embodiment the method further comprises: I) measuring the biomarkers in the subject before and after administration of a putative neuroprotective agent; and II) determining changes in amounts of proteins or patterns of biomarkers, wherein changes toward normal amounts or patterns indicate efficacy of the neuroprotective agent. In another embodiment the method further comprises: measuring the biomarkers in the subject at two different times; and determining changes in amounts of proteins or patterns of biomarkers, wherein changes indicate a change in a neurodegenerative state. In another embodiment the method comprises collecting a plurality of biological samples from the subject over a time period, optionally wherein the subject is receiving a putative or known neuroprotective agent during the time period, wherein the diagnostic algorithm predicts rates of disease progression or degree of response to the putative neuroprotective agent.

In another aspect provided herein is a method comprising: a) providing a dataset comprising, for each of a plurality of subjects, values indicating (1) state of a neurodegenerative condition, and (2) measures of a set of biomarkers, wherein the set of biomarkers includes: (i) at least one signaling kinase and, optionally, at least one oligomeric form of a neurodegenerative protein; or (ii) a plurality of different signaling kinases; and b) performing a statistical analysis on the dataset to develop a model that infers the state of the neurodegenerative condition in an individual. In one embodiment at least one of the signaling kinases is a kinase of the PI3K-Akt-mTOR signaling pathway. In another embodiment at least one of the signaling kinases is selected from mitogen-activated protein kinase (MAPK or MEK), extracellular signal-regulated kinases (ERK), glycogen synthase kinase 3 beta (GSK3B), AKT kinase and beclin. In another embodiment the neurodegenerative protein selected from alpha synuclein, amyloid beta, tau, or huntingtin. In another embodiment the oligomeric form of the neurodegenerative protein is a collection of oligomeric forms, e.g., oligomers of alpha synuclein, e.g., alpha synuclein 2-50, e.g., alpha synuclein 4-30, e.g., alpha synuclein 4-20. In another embodiment the statistical analysis is performed by computer. In another embodiment the statistical analysis is not performed by computer. In another embodiment the statistical analysis comprises: correlational, Pearson correlation, Spearman correlation, chi-square, comparison of means (e.g., paired T-test, independent T-test, ANOVA) regression analysis (e.g., simple regression, multiple regression, linear regression, non-linear regression, logistic regression, polynomial regression. stepwise regression, ridge regression, lasso regression, elasticnet regression) or non-parametric analysis (e.g., Wilcoxon rank-sum test, Wilcoxon sign-rank test, sign test). In another embodiment the statistical analysis comprises training a machine learning algorithm on the dataset. In another embodiment the machine learning algorithm is selected from: artificial neural networks (e.g., back propagation networks), decision trees (e.g., recursive partitioning processes, CART), random forests, discriminant analyses (e.g., Bayesian classifier or Fischer analysis), linear classifiers (e.g., multiple linear regression (MLR), partial least squares (PLS) regression, principal components regression (PCR)), mixed or random-effects models, non-parametric classifiers (e.g., k-nearest neighbors), support vector machines, and ensemble methods (e.g., bagging, boosting). In another embodiment the state is selected from diagnosis, stage, prognosis or progression of the neurodegenerative condition. In another embodiment the state is measured as a categorical variable (e.g., a binary state or one of a plurality of categorical states). In another embodiment the categories comprise a diagnosis consistent with (e.g., positive or diagnosed as having) having the neurodegenerative condition and inconsistent with (e.g., negative or diagnosed as not having) having the neurodegenerative condition. In another embodiment the categories comprise different stages of the neurodegenerative condition. In another embodiment the state is measured as a continuous variable (e.g., on a scale). In another embodiment the continuous variable is a range is or degrees of the neurodegenerative condition. In another embodiment the subjects are animals, e.g., fish, avians, amphibians, reptiles, or mammals, e.g., rodents, primates or humans. In another embodiment the plurality of subjects is at least any of 10, 25, 50, 100, 200, 400 or 800. In another embodiment, for each subject, the sample for which the quantitative measures are determined are taken at a first time point and the state of the neurodegenerative condition is determined at a second, later time point. In another embodiment the biological sample comprises blood or a blood fraction (e.g., plasma or serum). In another embodiment the neurodegenerative condition is a synucleinopathy, e.g., Parkinson's Disease or Lewy Body Dementia. In another embodiment the neurodegenerative condition is an amyloidopathy, e.g., Alzheimer's Disease, a tauopathy, e.g., Alzheimer's Disease or Huntington's disease.

In another aspect provided herein is a method of inferring a risk of developing, a diagnosis of, a stage of, a prognosis of or a progression of a neurodegenerative condition characterized by a neurodegenerative protein, wherein the method comprises: a) measuring, from a biological sample from a subject that is enriched for neuronally derived microparticles, e.g., exosomes, a set of biomarkers to create a dataset, wherein the set of biomarkers includes: (1) at least one signaling kinase and, optionally, at least one oligomeric form of a neurodegenerative protein; or (2) a plurality of different signaling kinases; and b) executing a model, e.g., a model as described herein, on the dataset to infer a risk of developing, a diagnosis of, a stage of, a prognosis of or a progression of the neurodegenerative condition. In one embodiment at least one of the signaling kinases is a kinase of the PI3K-Akt-mTOR signaling pathway. In another embodiment at least one of the signaling kinases is selected from mitogen-activated protein kinase (MAPK or MEK), extracellular signal-regulated kinases (ERK), glycogen synthase kinase 3 beta (GSK3B), AKT kinase and beclin. In another embodiment the neurodegenerative protein selected from alpha synuclein, amyloid beta, tau, or huntingtin. In another embodiment the oligomeric form of the neurodegenerative protein is a collection of oligomeric forms, e.g., oligomers of alpha synuclein, e.g., alpha synuclein 2-50, e.g., alpha synuclein 4-30, e.g., alpha synuclein 4-20. In another embodiment the neurodegenerative protein forms for which the quantitative measures are determined are selected from: (I) at least one oligomeric form; (II) a plurality of oligomeric forms; (Ill) at least one oligomeric form and at least one monomeric form; (IV) a plurality of oligomeric forms and at least one monomeric form; (V) at least one oligomeric form and a plurality of monomeric forms; and (VI) a plurality of oligomeric forms and a plurality of monomeric forms. In another embodiment at least one of the oligomeric forms comprises a collection of species of the neurodegenerative protein. In another embodiment the model comprises comparing relative amounts an oligomeric form to monomeric form of the neurodegenerative protein to relative amounts in a statistically significant number of control individuals. In another embodiment the model comprises detecting a pattern of relative amounts of a plurality of the oligomeric forms from which model the inference is made. In another embodiment the subject is asymptomatic or preclinical for a neurodegenerative condition. In another embodiment the subject presents to a healthcare provider, such as a doctor, during a routine office visit or as part of a doctor's ordinary practice of medicine. In another embodiment the model is executed by computer. In another embodiment the model is not executed by computer.

In another aspect provided herein is a method for determining effectiveness of a therapeutic intervention in treating a neurodegenerative condition, wherein the method comprises: (a) inferring, in each subject in a population comprising a plurality of subjects, an initial state of a neurodegenerative condition by: (1) measuring, from a biological sample from a subject that is enriched for neuronally derived microparticles, e.g., exosomes, a set of biomarkers to create a dataset, wherein the set of biomarkers includes: (i) at least one signaling kinase and, optionally, at least one oligomeric form of a neurodegenerative protein; or (ii) a plurality of different signaling kinases; and (2) inferring the initial state using a model, e.g., a model as described herein; (b) after inferring, administering the therapeutic intervention to the subjects; (c) after administering, inferring, in each subject individual in the population, a subsequent a subsequent state of the neurodegenerative condition by: (1) measuring, from a biological sample from a subject that is enriched for neuronally derived microparticles, e.g., exosomes, a set of biomarkers to create a dataset, wherein the set of biomarkers includes; (i) at least one signaling kinase and, optionally, at least one oligomeric form of a neurodegenerative protein; or (ii) a plurality of different signaling kinases; and (2) inferring the subsequent state using the model; and (d) based on the initial and subsequent inferences in the population, determining that the therapeutic intervention is effective if the subsequent inferences exhibit a statistically significant change toward a normal state compared with the initial inferences, or that the therapeutic intervention is not effective if the subsequent inferences do not exhibit a statistically significant change compared with the initial inferences toward a normal state. In another embodiment at least one of the signaling kinases is a kinase of the PI3K-Akt-mTOR signaling pathway. In another embodiment at least one of the signaling kinases is selected from mitogen-activated protein kinase (MAPK or MEK), extracellular signal-regulated kinases (ERK), glycogen synthase kinase 3 beta (GSK3B), AKT kinase and beclin. In another embodiment the neurodegenerative protein selected from alpha synuclein, amyloid beta, tau, or huntingtin. In another embodiment the oligomeric form of the neurodegenerative protein is a collection of oligomeric forms, e.g., oligomers of alpha synuclein, e.g., alpha synuclein 2-50, e.g., alpha synuclein 4-30, e.g., alpha synuclein 4-20. In another embodiment the therapeutic intervention comprises administration of a drug or combination of drugs. In another embodiment the population comprises at least 20, at least 50, at least 100 or at least 200 subjects, wherein at least 20%, at least 35%, at least 50%, or at least 75% of the subjects initially have elevated relative amounts of oligomeric forms of the protein to monomeric forms of the protein. In another embodiment at least 20%, at least 25%, at least 30%, or at least 35%, least 50%, at least 66%, at least 80%, or 100% of the subjects initially have a diagnosis of a neurodegenerative condition. In another embodiment the inference is made by computer. In another embodiment the inference is not made by computer.

In another aspect provided herein is a method for qualifying subjects for a clinical trial of a therapeutic intervention for the treatment or prevention of a neurodegenerative condition comprising: a) determining that a subject is abnormal with respect with a neurodegenerative condition by: (1) measuring, from a biological sample from a subject that is enriched for neuronally derived microparticles, e.g., exosomes, a set of biomarkers to create a dataset, wherein the set of biomarkers includes; (i) at least one signaling kinase and, optionally, at least one oligomeric form of a neurodegenerative protein; or (ii) a plurality of different signaling kinases; (2) executing a model, e.g., a model as described herein, on the profile to infer that the subject is abnormal with respect with the neurodegenerative condition; and b) enrolling the subject in the clinical trial of a potentially therapeutic intervention for said neurodegenerative condition. In one embodiment at least one of the signaling kinases is a kinase of the PI3K-Akt-mTOR signaling pathway. In another embodiment at least one of the signaling kinases is selected from mitogen-activated protein kinase (MAPK or MEK), extracellular signal-regulated kinases (ERK), glycogen synthase kinase 3 beta (GSK3B), AKT kinase and beclin. In another embodiment the neurodegenerative protein is selected from alpha synuclein, amyloid beta, tau, or huntingtin. In another embodiment the oligomeric form of the neurodegenerative protein is a collection of oligomeric forms, e.g., oligomers of alpha synuclein, e.g., alpha synuclein 2-50, e.g., alpha synuclein 4-30, e.g., alpha synuclein 4-20. In another embodiment the model is executed by computer. In another embodiment the model is not executed by computer.

In another aspect provided herein is a method of monitoring progress of a subject on a therapeutic intervention for a neurodegenerative condition comprising: (a) inferring, in the subject, an initial state of a neurodegenerative condition by: (1) determining, from a biological sample from a subject that is enriched for neuronally derived microparticles, e.g., exosomes, measures of a set of biomarkers, wherein the set of biomarkers includes: (i) at least one signaling kinase and, optionally, at least one oligomeric form of a neurodegenerative protein; or (ii) a plurality of different signaling kinases; and (2) executing a model, e.g., a model as described herein, to infer an initial state of the neurodegenerative condition; (b) after inferring, administering the therapeutic intervention to the subject; (c) after administering, inferring, in the subject, a subsequent state of the neurodegenerative condition by: (1) determining, from a biological sample from a subject that is enriched for neuronally derived microsomal particles, a biomarker profile comprising amounts of each of a plurality of different signaling kinases to create a dataset; and (2) executing a model, e.g. a model as described herein, to infer a subsequent state of the neurodegenerative condition; (d) based on the initial and subsequent state inferences, determining that the subject is responding positively to the therapeutic intervention if the subsequent inference exhibits a change toward a normal state compared with the initial inferences, or that the therapeutic intervention is not effective if the subsequent inferences do not exhibit a change compared with the initial inferences toward a normal state. In one embodiment at least one of the signaling kinases is a kinase of the PI3K-Akt-mTOR signaling pathway. In another embodiment at least one of the signaling kinases is selected from mitogen-activated protein kinase (MAPK or MEK), extracellular signal-regulated kinases (ERK), glycogen synthase kinase 3 beta (GSK3B), AKT kinase and beclin. In another embodiment the neurodegenerative protein selected from alpha synuclein, amyloid beta, tau, or huntingtin. In another embodiment the oligomeric form of the neurodegenerative protein is a collection of oligomeric forms, e.g., oligomers of alpha synuclein, e.g., alpha synuclein 2-50, e.g., alpha synuclein 4-30, e.g., alpha synuclein 4-20. In another embodiment wherein the model is executed by computer. In another embodiment the model is not executed by computer.

In another aspect provided herein is a method comprising: (a) determining, by the method as disclosed herein, that a subject has a neurodegenerative condition, and (b) administering to the subject a palliative or neuroprotective therapeutic intervention efficacious to treat the condition. In one embodiment the therapeutic intervention moves a biomarker profile of the subject toward normal, wherein a movement toward normal indicates neuroprotection.

In another aspect provided herein is a method comprising administering to a subject determined by the method as disclosed herein, to have an abnormal pattern of biomarkers, a palliative or neuroprotective therapeutic intervention effective to treat the condition. In one embodiment the subject is asymptomatic or preclinical for the neurodegenerative condition.

In another aspect provided herein is a kit comprising reagents sufficient to detect either: (1) at least one of signaling kinase and at least one oligomeric form of a neurodegenerative protein; or (2) a plurality of different signaling kinases. In one embodiment the reagents comprise antibodies.

In another aspect provided herein is a method of inferring a risk of developing, a diagnosis of, a stage of, a prognosis of or a progression of a neurodegenerative condition, wherein the method comprises: a) measuring, from a biological sample from a subject that is enriched for neuronally derived microparticles, e.g., exosomes, a set of biomarkers to create a dataset, wherein the set of biomarkers includes: (1) at least one signaling kinase and, optionally, at least one oligomeric form of a neurodegenerative protein; or (2) a plurality of different signaling kinases; and b) correlating the dataset with a risk of developing, a diagnosis of, a stage of, a prognosis of or a progression of the neurodegenerative condition. In one embodiment at least one of the signaling kinases is a kinase of the PI3K-Akt-mTOR signaling pathway. In another embodiment at least one of the signaling kinases is selected from mitogen-activated protein kinase (MAPK or MEK), extracellular signal-regulated kinases (ERK), glycogen synthase kinase 3 beta (GSK3B), AKT kinase and beclin. In another embodiment wherein the neurodegenerative protein selected from alpha synuclein, amyloid beta, tau, or huntingtin. In another embodiment the oligomeric form of the neurodegenerative protein is a collection of oligomeric forms, e.g., oligomers of alpha synuclein, e.g., alpha synuclein 2-50, e.g., alpha synuclein 4-30, e.g., alpha synuclein 4-20.

In another aspect provided herein is a method comprising: (a) identifying a subject having a neurodegenerative condition or likely to positively respond to a treatment for a neurodegenerative condition, wherein identifying comprises: (1) measuring, in a sample from the subject enriched for neuronally derived exosomes (e.g., from the internal contents of the exosomes), a set of biomarkers, to create a biomarker profile, wherein the set of biomarkers includes one or a plurality of signaling kinases and, optionally, at least one oligomeric form of a neurodegenerative protein; and (2) determining, based on an abnormal biomarker profile, that the subject suffers from the neurodegenerative condition; and (b) administering to the identified subject, an effective amount of a pharmaceutical composition to treat the neurodegenerative condition. In one embodiment the neurodegenerative condition is a synucleopathic condition, and the pharmaceutical composition comprises comprising a dopamine agonist (e.g., pramipexole (e.g., Mirapex™), ropinirole (e.g., Requip), rotigotine (e.g., Neupro), apomorphine (e.g., Apokyn)), levodopa, carbidopa-levodopa (e.g., Rytary, Sinemet), a MAO-B inhibitor (e.g., selegiline (e.g., Eldepryl, Zelapar) or rasagiline (e.g., Azilect)), a catechol-O-methyltransferase (COMT) inhibitor (e.g., entacapone (Comtan) or tolcapone (Tasmar)), an anticholinergic (e.g., benztropine (e.g., Cogentin) or trihexyphenidyl), amantadine or a cholinesterase inhibitor (e.g., rivastigmine (Exelon)). In another embodiment the synucleopathic condition is Parkinson's Disease. In another embodiment the pharmaceutical composition comprises a dopamjne agonist. In another embodiment the pharmaceutical composition further comprises an NK1-antagogonist. In another embodiment the dopamine agonist is 6-propylamino-4,5,6,7-tetrahydro-1,3-benzothiazole-2-amine and the NK1-antagonist is aprepitant or rolapitant. In another embodiment the pharmaceutical composition further comprises an 5HT3-antagonist. In another embodiment the dopamine agonist is 6-propylamino-4,5,6,7-tetrahydro-1,3-benzothiazole-2-amine and the 5HT3 antagonist is ondansetron hydrochloride dihydrate.

In another aspect provided herein is a method comprising administering to a subject characterized as having a biomarker profile indicative of a neurodegenerative condition or being likely to positively respond to a treatment for a neurodegenerative condition, an effective amount of a pharmaceutical composition to treat the neurodegenerative condition; wherein the biomarker panel comprises set of biomarkers includes one or a plurality of signaling kinases and, optionally, at least one oligomeric form of a neurodegenerative protein measured from a sample from the subject enriched for neuronally derived exosomes (e.g., from the internal contents of the exosomes). In on embodiment In another embodiment the neurodegenerative condition is Parkinson's Disease, and wherein the pharmaceutical composition comprises a dopamine agonist.

In certain embodiments, the biomarkers are selected from one or a plurality of different signaling kinases and, optionally, one or a plurality of monomeric and/or oligomeric forms of each of one or a plurality of neurodegenerative proteins.

Other objects of the disclosure may be apparent to one skilled in the art upon reading the following specification and claims.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows a flow diagram of an exemplary method detecting kinases and, optionally, neurodegenerative protein forms from exosomes.

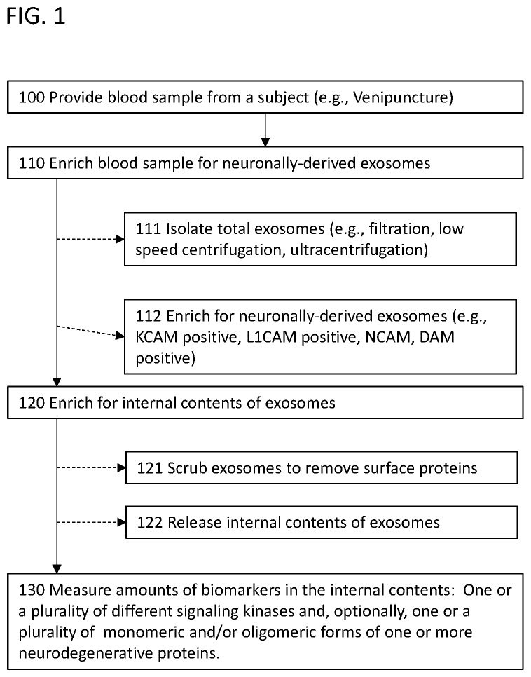

FIG. 2 shows a flow diagram of an exemplary protocol to validate drug efficacy.

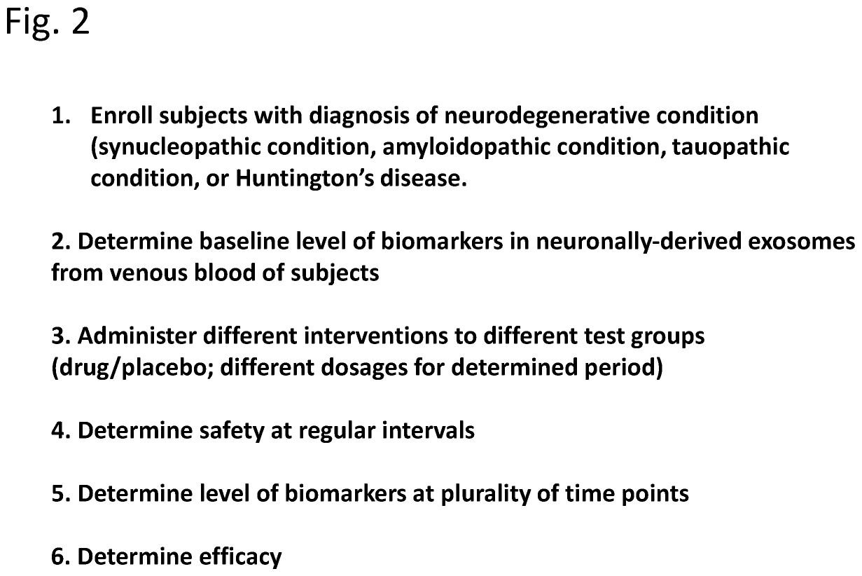

FIG. 3 shows an exemplary flow diagram of creating and validating a diagnostic model for diagnosing a neurodegenerative condition.

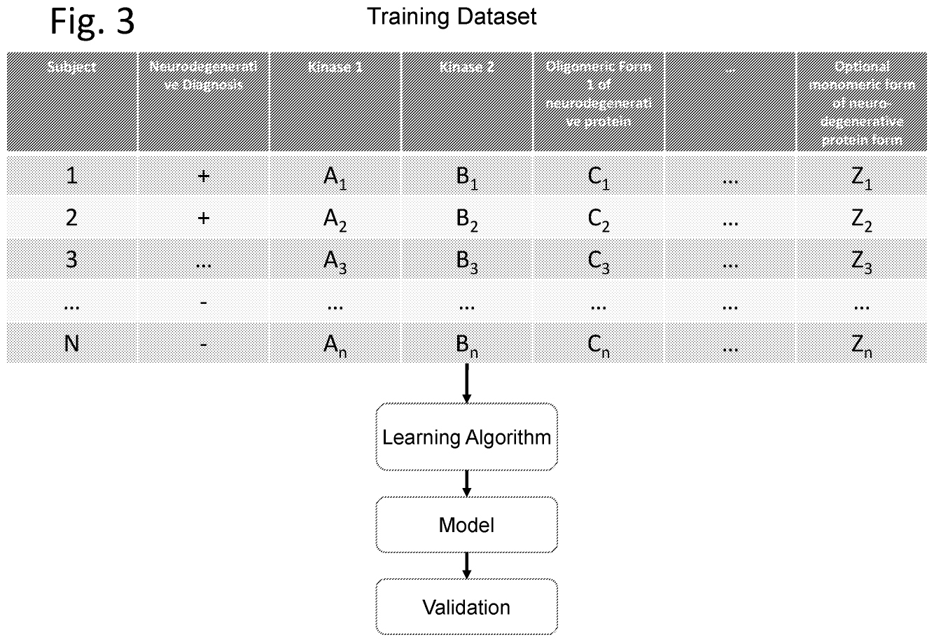

FIG. 4 shows an exemplary flow diagram for classifying a subject according to any of several states by executing a diagnostic algorithm, or model, on a biomarker profile.

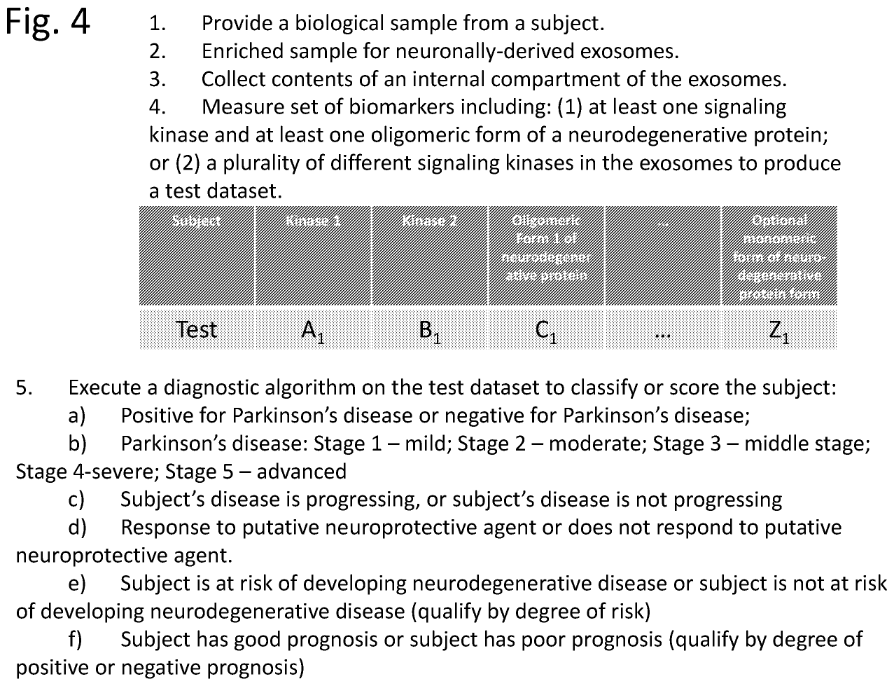

### DETAILED DESCRIPTION OF THE DISCLOSURE

### I. Biomarkers for Neurodegenerative Conditions

Methods disclosed herein are useful for diagnosis of and drug development for a variety of neurodegenerative conditions. These include, without limitation, synucleinopathies (e.g., Parkinson's disease, Lewy body dementia, multiple system atrophy), amyloidopathies (e.g., Alzheimer's disease), tauopathies (e.g., Alzheimer's disease, Progressive supranuclear palsy, Corticobasal degeneration), and Huntington's disease.

A. Biomarkers and Biomarker Profiles

Biomarkers are analytes that are associated, positively or negatively, alone or in combination, with a particular condition. Analytes that can function as biomarkers include any biological molecule or organic or inorganic molecule that is detectable in a subject or a subject sample. Biological molecules that can serve as biomarkers include, without limitation, polypeptides and polynucleotides, including, for example, proteins and peptides.

As used herein, the term “biomarker profile” refers to data indicating a measure of each of one or a plurality of biomarkers. Biomarker profiles used in certain embodiments of the methods described herein include measures of activity of one or a plurality of different kinases. In certain embodiments, biomarker profiles can further comprise measures of one or more neurodegenerative protein forms. A biomarker profile is a form of a dataset that includes data about the biomarkers.

The term “biomarker profile” may also be used to refer to a particular pattern in the profile which a model infers to be associated with a diagnosis, stage, progression, rate, prognosis, drug responsiveness and risk of developing a neurodegenerative condition.

A measurement of a variable, such as kinase activity, can be any combination of numbers and words. A measure can be any scale, including nominal (e.g., name or category), ordinal (e.g., hierarchical order of categories), interval (distance between members of an order), ratio (interval compared to a meaningful “0”), or a cardinal number measurement that counts the number of things in a set. Measurements of a variable on a nominal scale indicate a name or category, such a “healthy” or “unhealthy”, “old” or “young”, “form 1” or “form 2”, “subject 1 . . . subject n,” etc. Measurements of a variable on an ordinal scale produce a ranking, such as “first”, “second”, “third”; or “youngest” to “oldest”, or order from most to least. Measurements on a ratio scale include, for example, any measure on a pre-defined scale, such as mass, signal strength, concentration, age, etc., as well as statistical measurements such as frequency, mean, median, standard deviation, or quantile. Measurements on a ratio scale can be relative amounts or normalized measures. For example, in one embodiment, a biomarker profile comprises a relative amount of a first and second signaling kinase. In another embodiment a biomarker profile comprises a ratio of amounts of two different biomarker proteins.

Abnormal profiles (e.g., abnormal absolute or relative amounts of various signaling kinases) indicate pathologic activity, (or a characteristic bodily response to a pathogenic process) and thus time to future clinical onset and subsequent rates of clinical progression. Moreover, return toward normal in biomarker profiles (e.g., reductions in absolute or relative amounts of signaling kinases and/or oligomeric forms of neurodegenerative proteins) reflects the efficacy of a candidate neuroprotective intervention. Accordingly, the biomarker profiles described herein are useful for determining efficacy of drug candidates for their neuroprotective effect. As a practical matter, they may be considered essential to the practical conduct of neuroprotective drug trial in view of savings in both time and cost as well as a definitive means to quantified efficacy against a pathogenic process rather than its clinical manifestations.

Accordingly, biomarker profiles function not only as a diagnostic of an existing pathological state but also as a sentinel of pathology before clinical onset, e.g., when a subject is pre-symptomatic or preclinical, e.g., has signs or symptoms that are insufficient for a diagnosis of disease. This is relevant since the relative success of neuroprotective treatments often appear related to their earliest possible administration. Further, it is believed that these biomarker profiles indicate the stage (e.g., rate of or cumulative amount of neuronal loss) of a neurodegenerative condition. Accordingly, determining biomarker profiles can be of critical importance for determining effectiveness of a treatment, for example, in clinical trials and, for therapeutic interventions believed to be effective for treating neurodegeneration including, e.g., synucleinopathy, amyloidopathy, tauopathy or Huntington's disease in the individual.

B. Signaling Kinases

These diseases are characterized by abnormal changes in the activity (increased or decreased) of particular signaling kinases. Measuring activity of these signaling kinases in a subject can be used for diagnosis, prognosis, patient progress, patient stratification and drug development and testing.

Kinases include any kinase involved in signaling pathway.

Kinases associated with Parkinson's disease or the administration of medications that influence of the symptoms of Parkinson's disease (e.g., pramipexole (6-propylamino-4,5,6,7-tetrahydro-1,3-benzothiazole-2-amine)) include, without limitation, mTOR (mechanistic target of rapamycin), mitogen-activated protein kinase (MAPK or MEK), extracellular signal-regulated kinases (ERK), glycogen synthase kinase 3 beta (GSK3B), AKT kinase and beclin Leucine-Rich Repeat Kinase 2 (LRRK2), members of the c-Jun N-Terminal Kinase Signaling Pathway (JNK) (MAPK serine-threonine kinases), and Phosphatase and Tensin Homolog (PTEN)-Induced Putative Kinase 1 (PINK1).

Kinases associated with Alzheimer's disease include, without limitation, Tau protein kinases such as proline-directed protein kinases (PDPK), protein kinases non-PDPK and tyrosine protein kinases (TPK).

Kinases associated with Huntington's disease include, without limitation, mitogen-activated protein kinase, MEK, ERK, JNK, IKK, cell division protein kinase 5 (CDK5), AKT, MKP1.

These diseases also share in common the accumulation of toxic oligomeric polypeptide species, and in some cases abnormally phosphorylated oligomeric or monomeric forms, and the ability to detect such forms in neuronally derived exosomes.

C. Neurodegenerative Proteins

As used herein, the term “neurodegenerative protein” refers to a protein which, in an oligomerized form, is associated with neurodegeneration. Neurodegenerative proteins include, without limitation, alpha-synuclein, tau, amyloid beta and huntingtin.

It is believed that certain oligomerized forms or abnormally phosphorylated forms of brain polypeptides underlie a variety of neurodegenerative conditions. This includes, for example, the roles of alpha-synuclein in syncucleinopathic conditions, amyloid beta in amyloidopathic conditions, tau in tauopathic conditions and huntingtin in Huntington's disease. In particular, current evidence suggests that α-synuclein oligomers can act as a toxic species in PD and other synucleinopathies. In certain embodiments, the oligomeric species detected is an abnormally phosphorylated species.

Forms of neurodegenerative proteins include, without limitation, (I) at least one oligomeric form; (II) a plurality of oligomeric forms in combination (e.g., all oligomeric forms or a subset of oligomeric forms measured together, e.g., alpha synuclein 2-14), (III) each of a plurality of different oligomeric forms; (IV) at least one oligomeric form and at least one monomeric form; (V) a plurality of oligomeric forms and at least one monomeric form; and (VI) at least one oligomeric form and a plurality of monomeric forms. Forms of neurodegenerative proteins can be used in models to infer, among other things, neurodegenerative conditions or progression toward neurodegenerative conditions, typically with one or more oligomeric forms included in a model indicating the presence and activity of the disease or progression towards the disease. This includes increasing relative amounts of oligomeric alpha-synuclein forms indicating the presence and activity of a synucleinopathy, or progression towards a synucleinopathy; increasing relative amounts of oligomeric amyloid beta indicating the presence and activity of an amyloidopathy, or progression towards an amyloidopathy, increasing relative amounts of oligomeric or abnormally phosphorylated tau indicating the presence and activity of a tauopathy, or progression towards a tauopathy, and increasing relative amounts of oligomeric huntingtin indicating the presence and activity of Huntington's disease, or progression towards Huntington's disease. Accordingly, an abnormal profile of such oligomers indicates a process of neurodegeneration.

Neurodegenerative proteins forms can include one or more oligomeric forms and, optionally, one or more monomeric forms. This includes amounts of species of oligomeric and, optionally, monomeric alpha-synuclein; oligomeric and, optionally, monomeric amyloid beta, oligomeric and, optionally hyperphosphorylated and, optionally, monomeric tau; and oligomeric and, optionally, monomeric huntingtin. For example, a biomarker profile can include (I) at least one oligomeric form; (II) a plurality of oligomeric forms; (Ill) at least one oligomeric form and at least one monomeric form; (IV) a plurality of oligomeric forms and at least one monomeric form; (V) at least one oligomeric form and a plurality of monomeric forms; and (VI) a plurality of oligomeric forms and a plurality of monomeric forms.

Protein forms can refer to individual protein species or collections of species. For example, a 6-mer of alpha-synuclein is a form of alpha backspace-synuclein. Also, the collection of 6-mers to 18-mers of alpha-synuclein, collectively, can be a form of alpha-synuclein.

A biomarker profile can include a plurality of forms of a protein. In one embodiment, a biomarker profile can include quantitative measures of each of a plurality of oligomeric forms and monomeric form of the neurodegenerative protein. So, for example, the biomarker profile could include quantitative measures of each of a dimer, trimer, tetramer, 5-mer, 6-mer, 7-mer, 8-mer, 9-mer, 10-mer, 11-mer, 12-mer, 13-mer, 14-mer, 15-mer, 16-mer, 19-mer, 20-mer, 24-mer, 50-mer, etc.

As used herein, a “synuclein biomarker profile” refers to a profile comprising oligomeric and, optionally, monomeric alpha-synuclein, the term “amyloid biomarker profile” refers to a profile comprising oligomeric and, optionally, monomeric beta-amyloid, the term “tau biomarker profile” refers to a profile comprising oligomeric and, optionally, monomeric tau, the term “huntingtin biomarker profile” refers to a profile comprising oligomeric and, optionally, monomeric huntingtin.

As used herein, the term “monomeric protein/polypeptide” refers to a single, non-aggregated protein or polypeptide molecule, including any species thereof, such as phosphorylated species. As used herein, the term “oligomeric protein/polypeptide” refers to individual oligomeric species or an aggregate comprising a plurality of oligomeric species, including phosphorylated species. It is understood that measurement of an oligomeric form of a protein, as used herein, can refer to measurement of all oligomeric forms (total oligomeric form) or specified oligomeric forms. Specified oligomeric forms can include, for example, forms within a particular size range or physical condition such as for example soluble fibrils.

In each of these conditions, it is believed that oligomerized/aggregated forms of polypeptides described herein are toxic to neurons in that the biomarker profiles comprising oligomeric forms and, optionally, monomeric forms of these polypeptides function in models to infer pathologic activity. In particular, increased relative amounts of oligomeric forms as compared with monomeric forms indicate pathology. Measures of these biomarkers can be used to track subject responses to therapies that are either in existence or in development as well as to predict development of disease or the state or progress of existing disease.

### II. Neurodegenerative Conditions and Associated Proteins

A. Synucleinopathies

1. Conditions

As used herein, the terms “synucleinopathy” and “syncucleinopathic condition” refer to a condition characterized by abnormal profiles of oligomeric alpha-synuclein, which is an abnormal, aggregated form of alpha-synuclein. In certain embodiments, synucleinopathies manifest as clinically evident syncucleinopathic disease such as, for example, PD, Lewy body dementia, multiple system atrophy and some forms of Alzheimer's disease, as well as other rare neurodegenerative disorders such as various neuroaxonal dystrophies. Signs and, optionally, symptoms sufficient for a clinical diagnosis of a syncucleinopathic disease are those generally sufficient for a person skilled in the art of diagnosing such conditions to make such a clinical diagnosis.

Parkinson's disease (“PD”) is a progressive disorder of the central nervous system (CNS) with a prevalence of 1% to 2% in the adult population over 60 years of age. PD is characterized by motor symptoms, including tremor, rigidity, postural instability and slowness of voluntary movement. The cause of the idiopathic form of the disease, which constitutes more than 90% of total PD cases, remains elusive, but is now considered to involve both environmental and genetic factors. Motor symptoms are clearly related to a progressive degeneration of dopamine-producing neurons in the substantia nigra. More recently, PD has become recognized one of a group of multi-system disorders, which mainly affect the basal ganglia (e.g., PD), or the cerebral cortex (e.g., Lewy body dementia), or the basal ganglia, brain stem and spinal cord (e.g., multiple system atrophy) and which are all linked by the presence of intracellular deposits (Lewy bodies) consisting mainly of a brain protein called alpha-synuclein. Accordingly, these disorders, along with Hallevorden-Spatz syndrome, neuronal axonal dystrophy, and traumatic brain injury have often been termed “Synucleinopathies”.

Signs and symptoms of PD may include, for example, tremors at rest, rigidity, bradykinesia, postural instability and a festinating parkinsonian gate. One sign of PD is a positive response in these motor dysfunctions to carbidopa-levodopa.

Clinically recognized stages of Parkinson's disease include the following: Stage 1—mild; Stage 2—moderate; Stage 3—middle stage; Stage 4—severe; Stage 5—advanced.

Pramipexole (sold under the brand name Mirapex™) is a drug that may treat idiopathic Parkinsonism. Pramipexole has activity as an extracellular signal-regulated kinase (ERK) agonist. Accordingly, determining the effect of pramipexole, and other kinase modulators, on kinase activity is useful in determining effectiveness of the drug on Parkinson's Disease.

At present, the diagnosis of PD mainly rests on the results of a physical examination that is often quantified by the use of the modified Hoehn and Yahr staging scale (Hoehn and Yahr, 1967, Neurology, 17:5, 427-442) and the Unified Parkinson's Disease Rating Scale (UPDRS). The differential diagnosis of PD vs. other forms of parkinsonism, e.g., progressive supranuclear palsy (PSP), can prove difficult and misdiagnosis can thus occur in up to 25% of patients. Indeed, PD generally remains undetected for years before the initial clinical diagnosis can be made. When this happens, the loss of dopamine neurons in the substantia nigra already exceeds 50% and may approach 70%. No blood test for PD or any related synucleinopathy has yet been validated. While imaging studies using positron emission tomography (PET) or MRI have been used in the diagnosis of PD by providing information about the location and extent of the neurodegenerative process, they confer little or no information about the pathogenesis of the observed degeneration and do not guide the selection of a particular syncucleinopathic-specific intervention.

Lewy body dementias (LBD) affect about 1.3 million people in the US. Symptoms include, for example, dementia, cognitive fluctuations, parkinsonism, sleep disturbances and hallucinations. It is the second most common form of dementia after Alzheimer's disease and usually develops after the age of 50. Like Parkinson's disease, LBD is characterized by abnormal deposits of alpha-synuclein in the brain.

Multiple system atrophy (MSA) is classified into two types, Parkinsonian type and cerebellar type. The parkinsonian type is characterized by, for example, parkinsonian symptoms of PD. The cerebellar type is characterized by, for example, impaired movement and coordination, dysarthria, visual disturbances and dysphagia. MSA symptoms reflect cell loss and gliosis or a proliferation of astrocytes in damaged areas of brain, especially the substantia nigra, striatum, inferior olivary nucleus, and cerebellum. Abnormal alpha-synuclein deposits are characteristic.

Diagnostic error rates for PD and other synucleinopathies can be relatively high, especially at their initial stages, a situation that could become important with the introduction of effective disease modifying therapies, such as neuroprotective therapies.

2. Alpha-Synuclein

Alpha-synuclein is a protein found in the human brain. The human alpha-synuclein protein is made of 140 amino acids and is encoded by the SNCA gene (also called PARK1). (Alpha-synuclein: Gene ID: 6622; Homo sapiens; Cytogenetic Location: 4q22.1.)

As used herein, the term “alpha-synuclein” includes normal (unmodified) species, as well as modified species. Alpha-synuclein can exist in monomeric or aggregated forms. Alpha-synuclein monomers can aberrantly aggregate into oligomers, and oligomeric alpha-synuclein can aggregate into fibrils. Fibrils can further aggregate to form intracellular deposits called Lewy bodies. It is believed that monomeric alpha-synuclein and its various oligomers exist in equilibrium. Alpha-synuclein processing in brain can also produce other putatively abnormal species, such as alpha-synuclein phosphorylated at serine 129 (“p129 alpha-synuclein”).

Alpha-synuclein is abundantly expressed in human central nervous system (CNS) and to a lesser extent in various other organs. In brain, alpha-synuclein is mainly found in neuronal terminals, especially in the cerebral cortex, hippocampus, substantia nigra and cerebellum, where it contributes to the regulation of neurotransmitter release. Under normal circumstances, this soluble monomeric protein tends to form a stably folded tetramer that resists aggregation. But, in certain pathological conditions, for unknown reasons, the alpha-synuclein abnormally beta pleats, misfolds, oligomerizes and aggregates to eventually form fibrils, a metabolic pathway capable of yielding highly cytotoxic intermediates.

As used herein, the term “monomeric alpha-synuclein” refers to a single, non-aggregated alpha-synuclein molecule, including any species thereof. As used herein, the term “oligomeric alpha-synuclein” refers to an aggregate comprising a plurality of alpha-synuclein protein molecules. This includes total oligomeric alpha-synuclein and forms or selected species thereof. Oligomeric alpha-synuclein includes forms having at least two monomeric units up to protofibril forms. This includes oligomeric forms having, e.g., between 2 and about 100 monomeric units, e.g., between 4 and 16 monomeric units or at least 2, 3, 4 or 5 dozen monomeric units. As used herein, the term “relatively low weight synuclein oligomer” refers to synuclein oligomers comprised of up to 30 monomeric units (30-mers). Typically, relatively low weight synuclein oligomers are soluble. In certain embodiments, alpha-synuclein refers to the form or forms detected by the particular method of detection. For example, the forms can be those detectable with antibodies raised against particular monomeric or oligomeric forms of alpha-synuclein.

The neurotoxic potential of the aberrantly processed alpha-synuclein into oligomerized forms is now believed to contribute to the onset and subsequent progression of symptoms of the aforementioned pathological conditions, notably PD, Lewy body dementia, multiple system atrophy, and several other disorders. These are generally defined as a group of neurodegenerative disorders characterized in part by the intracellular accumulation of abnormal alpha-synuclein aggregates, some of which appear toxic and may contribute to the pathogenesis of the aforementioned disorders. Precisely how certain oligomerized forms of alpha-synuclein might cause neurodegeneration is not yet known, although a role for such factors as oxidative stress, mitochondrial injury, and pore formation has been suggested. Nevertheless, many now believe that processes leading to alpha-synuclein oligomerization and aggregation may be central to the cellular injury and destruction occurring in these disorders.

Some studies have shown that prefibrillar synuclein oligomers and protofibrils are especially prone to confer neurotoxicity (Loov et al., “α-Synuclein in Extracellular Vesicles: Functional Implications and Diagnostic Opportunities”, M. Cell Mol Neurobiol. 2016 April; 36(3):437-48. doi: 10.1007/s10571-015-0317-0.) Others suggest that lower order oligomeric synuclein species may be primarily responsible, and it remains hardly clear precisely which synuclein species, or which ensemble of species with differing beta-sheet arrangements, acting alone or in concert by a single or multiple pathologic mechanisms, is most neurotoxic in PD or in any related synucleinopathy (Wong et al., “α-synuclein toxicity in neurodegeneration: mechanism and therapeutic strategies”, Nat Med. 2017 Feb. 7; 23(2):1-13. doi: 10.1038/nm.4269).

A portion of intracellular synuclein, along with certain of its metabolic products, is packaged within exosomal vesicles and released into the intracellular fluid in brain from where it passes into the cerebrospinal fluid (CSF) and peripheral blood circulation. Alpha-synuclein is a protein found in the human brain. The human alpha-synuclein protein is made of 140 amino acids and is encoded by the SNCA gene (also called PARK1). (Alpha-synuclein: Gene ID: 6622; Homo sapiens; Cytogenetic Location: 4q22.1.)

B. Amyloidopathies

1. Conditions

As used herein, the term “amyloidopathy” refers to a condition characterized by accumulation of amyloid polymers in the brain. Amyloidopathies include, without limitation, Alzheimer's disease and certain other neurodegenerative disorders such as late stage PD. Alzheimer's Disease is the most prevalent form of dementia. It is characterized at an anatomical level by the accumulation of amyloid plaques made of aggregated forms of beta-amyloid, as well as neurofibrillary tangles. Symptomatically is characterized by progressive memory loss, cognitive decline and neurobehavioral changes. Alzheimer's is progressive and currently there is no known way to halt or reverse the disease.

2. Amyloid beta

Amyloid beta (also called amyloid-β, Aβ, A-beta and beta-amyloid) is a peptide fragment of amyloid precursor protein. Amyloid beta typically has between 36 and 43 amino acids. Amyloid beta aggregates to form soluble oligomers which may exist in several forms. It is believed that misfolded oligomers of amyloid beta can cause other amyloid beta molecules to assume a mis-folded oligomeric form. A-beta₁₋₄₂ has the amino acid sequence: DAEFRHDSGY EVHHQKLVFF AEDVGSNKGA IIGLMVGGW IA [SEQ ID NO: 1].

In Alzheimer's disease, amyloid-β and tau proteins become oligomerized and accumulate in brain tissue where they have appear to cause neuronal injury and loss; indeed, some aver that such soluble intermediates of aggregation, or oligomers, are the key species that mediate toxicity and underlie seeding and spreading in disease (The Amyloid-β Oligomer Hypothesis: Beginning of the Third Decade. Cline E N, Bicca M A, Viola K L, Klein W L. J Alzheimers Dis. 2018; 64(s1):5567-5610; “Crucial role of protein oligomerization in the pathogenesis of Alzheimer's and Parkinson's diseases,” Choi M L, Gandhi S. FEBS J. 2018 Jun. 20.) Amyloid β oligomers are crucial for the onset and progression of AD and represent a popular drug target, being presumably the most direct biomarker. Tau protein may also become abnormally hyperphosphorylated.

Methods in current use to quantify monomeric and oligomeric forms of A-beta include enzyme linked immunosorbent assays (ELISA), methods for single oligomer detection, and others, which are mainly biosensor-based methods. (“Methods for the Specific Detection and Quantitation of Amyloid-β Oligomers in Cerebrospinal Fluid”, Schuster J, Funke S A. J Alzheimers Dis. 2016 May 7; 53(1):53-67.)

The surface-based fluorescence intensity distribution analysis (sFIDA) features both highly specific and sensitive oligomer quantitation as well as total insensitivity towards monomers (“Advancements of the sFIDA method for oligomer-based diagnostics of neurodegenerative diseases”, Kulawik A. et al., FEBS Lett. 2018 February; 592(4):516-534).

C. Tauopathies

1. Conditions

As used herein, the term “tauopathy” refers to a condition characterized by accumulation of and aggregation of in association with neurodegeneration. Tauopathies include, without limitation, Alzheimer's disease (“AD”), progressive supranuclear palsy, corticobasal degeneration, frontotemporal dementia with parkinsonism-linked to chromosome 17, and Pick disease.

AD is also characterized by a second pathological hallmark, the neurofibrillary tangle (NFT). NFTs are anatomically associated with neuronal loss, linking the process of NFT formation to neuronal injury and brain dysfunction. The main component of the NFT is a hyperphosphorylated form of tau, a microtubule-associated protein. During NFT formation, tau forms a variety of different aggregation species, including tau oligomers. Increasing evidence indicates that tau oligomer formation precedes the appearance of neurofibrillary tangles and contributes importantly to neuronal loss. (J Alzheimers Dis. 2013; 37(3):565-8 “Tauopathies and tau oligomers”, Takashima A.)

Nonfibrillar, soluble multimers appear to be more toxic than neurofibrillary tangles made up of filamentous tau.

In frontotemporal lobe dementia, full-length TAR DNA Binding Protein (“TDP-43”) forms toxic amyloid oligomers that accumulate in frontal brain regions. TDP-43 proteinopathies, which also include amyotrophic lateral sclerosis (ALS), are characterized by inclusion bodies formed by polyubiquitinated and hyperphosphorylated full-length and truncated TDP-43. The recombinant full-length human TDP-43 forms structurally stable, spherical oligomers that share common epitopes with an anti-amyloid oligomer-specific antibody. The TDP-43 oligomers have been found to be neurotoxic both in vitro and in vivo. (Nat Commun. 2014 Sep. 12; 5:4824. Full-length TDP-43 forms toxic amyloid oligomers that are present in frontotemporal lobar dementia-TDP patients). Determination of the presence and abundance of TDP-43 oligomers can be accomplished using a specific TDP-43 amyloid oligomer antibody called TDP-O among different subtypes of FTLD-TDP (“Detection of TDP-43 oligomers in frontotemporal lobar degeneration-TDP”, Kao P F, Ann Neurol. 2015 August; 78(2):211-21.)

2. Tau

Tau is a phosphoprotein with 79 potential Serine (Ser) and Threonine (Thr) phosphorylation sites on the longest tau isoform. Tau exists in six isoforms, distinguished by their number of binding domains. Three isoforms have three binding domains and the other three have four binding domains. The isoforms result from alternative splicing in exons 2, 3, and 10 of the tau gene. Tau is encoded by the MAPT gene, which has 11 exons. Haplogroup H1 appears to be associated with increased probability of certain dementias, such as Alzheimer's disease.

Various tau oligomeric species, including those ranging from 6- to 18-mers, have been implicated in the neurotoxic process associated with tauopathic brain disorders and measured by western blot and other techniques including single molecule fluorescence. (See, e.g., Kjaergaard M., et al., “Oligomer Diversity during the Aggregation of the Repeat Region of Tau” ACS Chem Neurosci. 2018 Jul. 17; Ghag G et al., “Soluble tau aggregates, not large fibrils, are the toxic species that display seeding and cross-seeding behavior”, Protein Sci. 2018 Aug. 20. doi: 10.1002/pro.3499; and Comerota M M et al., “Near Infrared Light Treatment Reduces Synaptic Levels of Toxic Tau Oligomers in Two Transgenic Mouse Models of Human Tauopathies”, Mol Neurobiol. 2018 Aug. 17.)

Methods to measure oligomeric tau species include immunoassay. Tau can be isolated by a common expression followed by chromatography, such as affinity, size-exclusion, and anion-exchange chromatography. This form can be used to immunize animals to generate antibodies. Aggregation of tau can be induced using arachidonic acid. Oligomers can be purified by centrifugation over a sucrose step gradient. Oligomeric forms of tau also can be used to immunize animals and generate antibodies. A sandwich enzyme-linked immunosorbent assay that utilizes the tau oligomer-specific TOC1 antibody can be used to detect oligomeric tau. The tau oligomer complex 1 (TOC1) antibody specifically identifies oligomeric tau species, in the tris insoluble, sarkosyl soluble fraction (Shirafuji N., et al, “Homocysteine Increases Tau Phosphorylation, Truncation and Oligomerization”, Int J Mol Sci. 2018 Mar. 17; 19(3).) (See, e.g., Methods Cell Biol. 2017; 141:45-64. doi: 10.1016/bs.mcb.2017.06.005. Epub 2017 Jul. 14. Production of recombinant tau oligomers in vitro. Combs B1, Tiernan CT1, Hamel C1, Kanaan NM.)

D. Huntington's Disease

1. Huntington's Disease

Huntington's disease is an inherited disease caused by an autosomal dominant mutation in the huntingtin gene. The mutation is characterized by duplication of CAG triplets. It is characterized by progressive neurodegeneration. Symptoms include movement disorders, such as involuntary movements, impaired gait and difficulty with swallowing and speech. It is also characterized by a progressive cognitive decline.

2. Huntingtin Protein

Huntington protein is encoded by the Huntington gene also called HTT or HD. The normal Huntington protein has about 3144 amino acids. The protein is normally about 300 KdA.

In Huntington's disease (HD), cleavage of the full-length mutant huntingtin (mHtt) protein into smaller, soluble aggregation-prone mHtt fragments appears to be a key process in the pathophysiology of this disorder. Indeed, aggregation and cytotoxicity of mutant proteins containing an expanded number of polyglutamine (polyQ) repeats is a hallmark of several diseases, in addition to HD. Within cells, mutant Huntingtin (mHtt) and other polyglutamine expansion mutant proteins exist as monomers, soluble oligomers, and insoluble inclusion bodies. (J Huntingtons Dis. 2012; 1(1):119-32. Detection of Mutant Huntingtin Aggregation Conformers and Modulation of SDS-Soluble Fibrillar Oligomers by Small Molecules. Sontag E M, et al., Brain Sci. 2014 Mar. 3; 4(1):91-122. Monomeric, oligomeric and polymeric proteins in Huntington disease and other diseases of polyglutamine expansion. Hoffner G. et al.) In certain embodiments, oligomers are 2-10 nm in height with an aspect ratio (longest distance across to shortest distance across) less than 2.5, indicating a globular structure.

### III. Detection and Measurement of Signaling Kinases and Neurodegenerative Proteins

A. Biological Samples

As used herein, the term “sample” refers to a composition comprising an analyte. A sample can be a raw sample, in which the analyte is mixed with other materials in its native form (e.g., a source material), a fractionated sample, in which an analyte is at least partially enriched, or a purified sample in which the analyte is at least substantially pure. As used herein, the term “biological sample” refers to a sample comprising biological material including, e.g., polypeptides, polynucleotides, polysaccharides, lipids and higher order levels of these materials such as, exosomes cells, tissues or organs.

As used herein, the term “microparticle” refers to an extracellular microvesicle or lipid raft protein aggregate having a hydrodynamic diameter of from about 50 to about 5000 nm. As such the term microparticle encompasses exosomes (about 50 to about 100 nm), microvesicles (about 100 to about 300 nm), ectosomes (about 50 to about 1000 nm), apoptotic bodies (about 50 to about 5000 nm) and lipid protein aggregates of the same dimensions. As used herein, the term “about” as used in reference to a value refers to 90 to 110% of that value. For instance, a diameter of about 1000 nm is a diameter within the range of 900 nm to 1100 nm.

Signaling kinases, as well as forms of neurodegenerative proteins, such as alpha-synuclein, amyloid beta, tau and huntingtin, can be detected in exosomes from bodily fluid samples from the subject. More particularly, isolates of neuronally derived exosomes are a preferred subset of exosomes for the detection and analysis of syncucleinopathic conditions. In particular, proteins from internal compartments of an exosome are useful.

Exosomes can be isolated from a variety of biological samples from a subject. In certain embodiments the biological sample is a bodily fluid. Bodily fluid sources of exosomes include, for example, blood (e.g., whole blood or a fraction thereof such as serum or plasma, e.g., peripheral venous blood), cerebrospinal fluid, saliva, milk and urine, or fractions thereof.

The use of venous blood as a source of exosomes is a preferred sample for a diagnostic test destined for use in both adults and children due to the safety, acceptability and convenience of routine venipuncture in medical settings. Because target analytes can be present in blood in small amounts, large samples may be taken. For example, a sample can have at least 5 ml, at least 10 ml at least 20 ml of blood. Serum can be prepared by allowing whole blood to clot and removing the clot by, e.g., centrifugation. Plasma can be prepared by, e.g., treating whole blood with an anti-coagulant, such as EDTA, and removal of blood cells by, e.g., centrifugation. The blood sample can be provided by taking the sample from a subject or by receiving the sample from a person who has taken blood from the subject. Blood samples typically will be stored cold, e.g., on ice or frozen at −80° C.

B. Methods of Measuring Signaling Kinases and Neurodegenerative Proteins

1. Signaling Kinases

Kinases convert ATP into ADP in the phosphorylation of substrates. Various assay types to measure kinase activity are known in the art.

a) Radioactive Scintillation

Radioactive Scintillation assays measure the incorporation of 32P into a substrate by a kinase.

b) FRET (Fluorescence Resonance Energy Transfer)

Certain of these assays use amounts of ATP or ADP as indicators of kinase activity. In one such assay, a sample being tested for kinase activity, a substrate for the kinase and ATP are combined. If the kinase is present, it will phosphorylate the substrate using ATP. The remaining ADP can be detected by various assays. One such assay is a FRET (Fluorescence Resonance Energy Transfer) assay in which ADP in the sample after reaction is tagged with one of a donor or acceptor fluorophore. An antibody that binds to ADP and that comprises the other fluorophore of the pair, i.e., an acceptor or donor fluorophore, is added to the mixture.

The antibody binds to ADP. Upon excitation, the donor fluorophore transfers energy to the acceptor fluorophore, which fluoresces and can be detected.

c) Immunodetection

In another assay, a specific kinase can be immunoprecipitated using an antibody specific for the kinase. The precipitated kinase is used in a phosphorylation reaction with a substrate of the kinase. The product of a kinase reaction is detected by Western blot.

d) Commercially Available Kinase Assays

Many kinase assays are commercially available. These include, for example, essays available from Promega (Promega.com), which are specific for a number of different kinases. Another example is the Adapta® Universal Kinase Assay System available from Thermo Fisher Scientific (ThermoFisher.com). PerkinElmer™ (PerkinElmer.com) commercializes the LANCE® kinase assay, which uses a fluorescently labeled substrate and a europium-labeled antiphospho antibody to recognize a phosphorylated product, which is detectable through FRET. Samdi Tech, Inc. (SamdiTech.com) commercializes label-free assays that use mass spectrometry.

2. Neurodegenerative Proteins

Monomeric and oligomeric forms of proteins can be detected by any methods known in the art including, without limitation, immunoassay (e.g., ELISA), mass spectrometry, size exclusion chromatography, Western blot and fluorescence-based methods (e.g., fluorescence spectroscopy or FRET) and proximity ligation assay.

In a Western blot, proteins in a mixture are separated by electrophoresis. Separated proteins are blotted onto a solid support, such as a nitrocellulose filter, typically by electroblotting. Blotted proteins can be detected either by direct binding with a binding agent against a synuclein oligomers, or by indirect binding in which, for example, the blot is contacted with a labeled primary antibody directed against α-synuclein oligomers, which is allowed to bind with the oligomer. Typically, the blot is washed, to remove unbound antibody. Then, the oligomeric forms are detected using a labeled antibody (typically referred to as a secondary antibody) directed against the primary antibody or a tag attached to the primary antibody.

Labels can include, for example, gold nanoparticles, latex beads, fluorescent molecules, luminescent proteins and enzymes that produce detectable products from a substrate. Tags can include, for example, biotin.

Alternatively, oligomeric species in a mixture can be separated from one another and subsequently detected. Oligomeric species in a mixture can be separated by several methods. In one method, species are separated by electrophoresis. This includes gel electrophoresis. Electrophoresis methods include polyacrylamide gel electrophoresis (“PAGE”) and agarose gel electrophoresis. In one method, native PAGE or blue native PAGE are used. Native PAGE Bis-Tris gels are available from, e.g., ThermoFisher®. In a method called packed-capillary electrophoresis, or “pCE”, arbitrarily wide pores are created by packing nonporous colloidal silica in capillaries. Alternatively, species can be separated by chromatography, such as size exclusion chromatography, liquid chromatography or gas chromatography.

Once separated, specific oligomeric forms of α-synuclein can be differentiated. This can be done without the need for binding agents that specifically bind to a particular oligomeric form because they are already separated and, therefore, distinguishable. A binding agent that binds to α-synuclein oligomers, in general, can be used to detect the forms. Their location on a gel, or time or elution from a column can be used to indicate the particular form detected. For example, larger oligomers typically migrate more slowly in a gel than smaller oligomers.

a) Alpha-Synuclein

Amounts of monomeric alpha-synuclein and oligomeric alpha-synuclein can be determined individually. Alternatively, total alpha-synuclein in the sample can be measured with either of monomeric alpha-synuclein or oligomeric alpha-synuclein and the amount of the other species can be determined based on the difference.

Monomeric, oligomeric and total alpha-synuclein can be detected by, for example, immunoassay (e.g., ELISA or Western blot), mass spectrometry or size exclusion chromatography. Antibodies against alpha-synuclein are commercially available from, for example, Abcam (Cambridge, MA), ThermoFisher (Waltham, MA) and Santa Cruz Biotechnology (Dallas, TX).

The following references described methods of measuring total alpha-synuclein content. Mollenhauer et al. (Movement Disorders, 32:8 p. 1117 (2017)) describes methods of measuring total alpha-synuclein from bodily fluids. Loov et al. (Cell Mol. Neurobiol., 36:437-448 (2016)) describes use of antibodies to isolate L1CAM positive exosomes from plasma. Abd-Elhadi et al. (Anal Bioanal Chem. (2016) November; 408(27):7669-72016) describes methods of determining total alpha-synuclein levels in human blood cells, CSF, and saliva determined by a lipid-ELISA.

Total alpha-synuclein can be detected in an ELISA using, for example, an anti-human α-syn monoclonal antibody 211 (Santa Cruz Biotechnology, USA) for capture and anti-human α-syn polyclonal antibody FL-140 (Santa Cruz Biotechnology, USA) for detection through a horseradish peroxidase (HRP)-linked chemiluminescence assay. Such an approach avoids detection of monomeric α-synuclein, but does not distinguish between the different multimeric forms.

Monomeric and oligomeric forms of alpha-synuclein can be detected by, for example, immunoassays using antibodies specific for the forms. See, e.g., Williams et al. (“Oligomeric alpha-synuclein and β-amyloid variants as potential biomarkers for Parkinson's and Alzheimer's diseases”, Eur J Neurosci. (2016) January; 43(1):3-16) and Majbour et al. (“Oligomeric and phosphorylated alpha-synuclein as potential CSF biomarkers for Parkinson's disease”, Molecular Neurodegeneration (2016) 11:7). El-Agnaf O. et al, (FASEB J. 2016; 20:419-425) described detection of oligomeric forms of alpha-synuclein protein in human plasma as a potential biomarker for PD.

Antibodies against alpha-synuclein monomers and oligomers can be produced by immunizing animals with alpha-synuclein monomers or oligomers. (See, e.g., U.S. Publications 2016/0199522 (Lannfelt et al.), 2012/0191652 (El-Agnaf). Alpha-synuclein oligomers can be prepared by the method of El Agnaf (U.S. 2014/0241987), in which freshly prepared α-synuclein solution was mixed with dopamine at 1:7 molar ratio (α-synuclein:dopamine) and incubated at 37° C. Antibodies against different oligomeric forms of alpha-synuclein are also described in Emadi et al. (“Isolation of a Human Single Chain Antibody Fragment Against Oligomeric α-Synuclein that Inhibits Aggregation and Prevents α-Synuclein-induced Toxicity”, J Mol Biol. 2007; 368:1132-1144. [PubMed: 17391701]) (dimers and tetramers) and Emadi et al. (“Detecting Morphologically Distinct Oligomeric Forms of α-Synuclein”, J Biol Chem. 2009; 284:11048-11058. [PubMed: 19141614]) (trimers and hexamers). Protofibril-binding antibodies are described in, for example, U.S. 2013/0309251 (Nordstrom et al.).

Monomeric alpha-synuclein and can be distinguished from polymeric alpha-synuclein by immunoassay using antibodies that are uniquely recognized by oligomeric forms of synuclein. Another method involves detection of mass differences, e.g., using mass spectrometry. Fluorescent methods can be used. (See, e.g., Sangeeta Nath, et al., “Early Aggregation Steps in α-Synuclein as Measured by FCS and FRET: Evidence for a Contagious Conformational Change” Biophys J. 2010 Apr. 7; 98(7): 1302-1311, doi: 10.1016/j.bpj.2009.12.4290; and Laura Tosatto et al., “Single-molecule FRET studies on alpha-synuclein oligomerization of Parkinson's disease genetically related mutants”, Scientific Reports 5, December 2015.) Another method involves measuring total alpha synuclein, followed by proteinase K digestion of non-pathological alpha synuclein and detection of remaining alpha synuclein. Another method involves an alpha synuclein proximity ligation assay. Protein ligation assay probes are generated from antibodies raised against the protein(s) of interest, one for each of the proteins involved in the putative interaction, which are conjugated to short oligonucleotides. If the probes bind interacting proteins, the oligonucleotides are sufficiently close to prime an amplification reaction, which can be detected by tagged oligonucleotides and observed as punctate signal, with each punctum representing an interaction. (Roberts R F et al., “Direct visualization of alpha-synuclein oligomers reveals previously undetected pathology in Parkinson's disease brain. Brain”, 2015; 138:1642-1657. doi: 10.1093/brain/awv040, and Nora Bengoa-Vergniory et al., “Alpha-synuclein oligomers: a new hope”, Acta Neuropathol. 2017; 134(6): 819-838).

The relative amount of oligomeric form of alpha-synuclein to monomers can be expressed as a ratio.

Quantity or amount can be expressed as a signal output from an assay or as an absolute amount after conversion, for example from a standard curve, e.g., in terms of mass per volume.

Alpha-synuclein species in the samples can be further stratified. For example, oligomers species can be divided into lower order oligomers, e.g., 2 to 24 monomeric units, higher order oligomers, e.g., 24 to 100 monomeric units, or protofibrils, etc.

### b) Amyloid Beta

Oligomers and monomers can be distinguished using an enzyme-linked immunosorbent assay (ELISA). This assay resembles a sandwich ELISA. The Aβ monomer contains one epitope, while oligomers contain a plurality these epitopes. Hence, if epitope-overlapping antibodies toward the above unique epitope were used for capturing and detecting antibodies, binding to a specific and unique epitope would generate competition between these two antibodies. In other words, the monomer would be occupied by the capturing or detection antibody but not by both. (“Oligomeric forms of amyloid-β protein in plasma as a potential blood-based biomarker for Alzheimer's disease”, Wang M J et al. Alzheimers Res Ther. 2017 Dec. 15; 9(1):98. “Potential fluid biomarkers for pathological brain changes in Alzheimer's disease: Implication for the screening of cognitive frailty”, Ruan Q et al., Mol Med Rep. 2016 October; 14(4):3184-98. “Methods for the Specific Detection and Quantitation of Amyloid-β Oligomers in Cerebrospinal Fluid,” Schuster J, Funke S A. J Alzheimers Dis. 2016 May 7; 53(1):53-67).

Oligomeric forms of amyloid beta for detection include, e.g. 4-24 mers of amyloid beta.

### c) Tau

Tau oligomers in biological fluids, e.g., CSF, can be measured by ELISA and Western blot analysis using anti-tau oligomer antibodies. (Sengupta U, et al., “Tau oligomers in cerebrospinal fluid in Alzheimer's disease”, Ann Clin Transl Neurol. 2017 April; 4(4): 226-235.

Oligomers of tau for detection include, e.g., low molecular weight oligomers, e.g., no more than 20-mers, e.g., 3-18 mers. The presence of soluble oligomers in the cerebral spinal fluid can be detected with monoclonal anti-oligomer antibodies with Western blot and Sandwich enzyme-linked immunosorbent assay (sELISA). David, M A et al., “Detection of protein aggregates in brain and cerebrospinal fluid derived from multiple sclerosis patients”, Front Neurol. 2014 Dec. 2; 5:251. Oligomeric forms of tau include hyperphosphorylated forms of oligomeric tau.

### d) Huntingtin

Recent quantification studies have made use of TR-FRET-based immunoassays. One detection method that combines size exclusion chromatography (SEC) and time-resolved fluorescence resonance energy transfer (TR-FRET) allows the resolution and definition of the formation, and aggregation of native soluble mHtt species and insoluble aggregates in brain. “Fragments of HdhQ150 mutant huntingtin form a soluble oligomer pool that declines with aggregate deposition upon aging”, Marcellin D. et al., PLoS One. 2012; 7(9):e44457.

A variety of published techniques have been used to assay oligomeric huntingtin species including, e.g., Agarose Gel Electrophoresis (AGE) analysis (under either native or mildly denaturing, 0.1% SDS conditions or Blue-Native PAGE under native conditions) which provides a number of immunoreactive oligomers; Anti-huntingtin antibodies differentially recognize specific huntingtin oligomers.

A one-step TR-FRET-based immunoassay has been developed to quantify soluble and aggregated mHtt in cell and tissue homogenates (TR-FRET-based duplex immunoassay reveals an inverse correlation of soluble and aggregated mutant huntingtin in Huntington's disease. Baldo B, et al., chem Biol. 2012 Feb. 24; 19(2):264-75).

Time-resolved Forster energy transfer (TR-FRET)-based assays represent high-throughput, homogeneous, sensitive immunoassays widely employed for the quantification of proteins of interest. TR-FRET is extremely sensitive to small distances and can therefore provide conformational information based on detection of exposure and relative position of epitopes present on the target protein as recognized by selective antibodies. We have previously reported TR-FRET assays to quantify HTT proteins based on the use of antibodies specific for different amino-terminal HTT epitopes (Fodale, V. et al., “Polyglutamine- and temperature-dependent conformational rigidity in mutant huntingtin revealed by immunoassays and circular dichroism spectroscopy”, PLoS One. 2014 Dec. 2; 9(12):e112262. doi: 10.1371/journal.pone.0112262. eCollection 2014.

### C. Isolation of Exosomes

Exosomes are extracellular vesicles that are thought to be released from cells upon fusion of an intermediate endocytic compartment, the multivesicular body (MVB), with the plasma membrane. The vesicles released in this process are referred to as exosomes. Exosomes are typically in the range of about 20 nm to about 100 nm.

Many methods of isolating exosomes are known in the art. These include, for example, immunoaffinity capture methods, size-based isolation methods, differential ultracentrifugation, exosome precipitation, and microfluidic-based isolation techniques. (Loov et al., “α-Synuclein in Extracellular Vesicles: Functional Implications and Diagnostic Opportunities”, M. Cell Mol Neurobiol. 2016 April; 36(3):437-48. doi: 10.1007/s10571-015-0317-0).

Amounts of exosomes in a sample can be determined by any of a number of methods. These include, for example, (a) immunoaffinity capture (IAC), (b) asymmetrical flow field-flow fractionation (AF4), (c) nanoparticle tracking analysis (NTA), (d) dynamic light scattering (DLS), and (e) surface plasmon resonance (SPR) [66]. Reprinted with permission from. Immunoaffinity capture (IAC) is the exosome capturing technology via immunoaffinity using an indirect isolation method. IAC quantifies exosomes by analyzing color, fluorescence, or electrochemical signals. Asymmetrical flow field-flow fractionation (AF4) separates and quantifies molecules using field-flow fraction and diffusion. Nanoparticle tracking analysis (NTA) separates and quantifies particles according to their size. NTA uses the rate of Brownian motion to analyze particles. This technique also tracks the concentration and size of exosomes using a light-scattering technique. Dynamic light scattering (DLS) determines particle size by light scattered by particles that exhibit Brownian motion. Surface plasmon resonance (SPR) is an immunoaffinity-based assay that captures exosomes with receptors on an SPR sensor surface. Binding changes the optical signals of receptors and their resonance can then be quantified through a light source. In another method, exosomes can be examined by electron microscopy, e.g., by visualizing at 120 kV in the Zeiss LSM 200 Transmission Electron Microscope.

### 1. Immunoaffinity Capture

Immunoaffinity capture methods use antibodies attached to an extraction moiety to bind exosomes and separates them from other materials in the sample. A solid support can be, for example, a magnetically attractable microparticle. Latex immunobeads can be used.

Qiagen describes its exoEasy Maxi Kit as using membrane affinity spin columns to efficiently isolate exosomes and other extracellular vesicles from serum, plasma, cell culture supernatant and other biological fluids.

### 2. Size-Based Methods

Size-based isolation methods include, for example, size exclusion chromatography and ultrafiltration. In size exclusion chromatography a porous stationary phase is used to separate exosomes based on size. In ultrafiltration, a porous membrane filter is used two separate exosomes based on their size or weight.

### 3. Differential Ultracentrifugation

Differential ultracentrifugation involves a series of centrifugation cycles of different centrifugal force and duration to isolate exosomes based on their density and size differences from other components in a sample. The centrifugal force can be, for example, from −100,000 to 120,000×g. Protease inhibitors can be used to prevent protein degradation. A prior cleanup step can be used to remove other large material from the sample.

### 4. Density Gradient Ultracentrifugation

Density gradient ultracentrifugation sorts exosomes using a gradient medium, such as such as sucrose, Nycodenz (iohexol), and iodixanol. Exosomes are isolated via ultracentrifugation to the layer in which the density of the gradient media is equal to that of the exosomes.

### 5. Polymer-Based Methods

Exosomes can be isolated from solutions of biological materials by altering their solubility or dispersibility. For example, addition of polymers such as polyethylene glycol (PEG), e.g., with a molecular weight of 8000 Da, can be used to precipitate exosomes from solution.

### 6. Microfluidic-Based Methods

Microfluidics-based methods can be used to isolate exosomes. These includes, for example, acoustic, electrophoretic and electromagnetic methods. For example, an acoustic nanofilter uses ultrasound standing waves to separate exosomes in a sample according to their size and density.

### 7. Other Methods

Other methods of isolating neuronally derived exosomes are described in, for example, Kanninnen, K M et al., “Exosomes as new diagnostic tools in CNS diseases”, Biochimica et Biophysica Acta, 1862 (2016) 403-410.

### 8. Enrichment for Neuronally-Derived Exosomes

Neuronally derived exosomes are exosomes produced by neurons. Preferably, the object of study is CNS-derived exosomes, that is, exosomes produced in the central nervous system, as distinguished from the peripheral nervous system. Methods described herein enrich a biological sample comprising exosomes for neuronally-derived exosomes and, by extension, CNS derived exosomes.

Immunoaffinity methods are useful for isolating neuronally derived exosomes using brain-specific biomarkers (e.g., neural and glial markers) one such marker is L1CAM. Another marker is KCAM. Other relatively brain-specific proteins can also serve in this capacity. neuronally derived exosomes are characterized by protein markers associated with the brain, including, for example, KCAM, L1CAM and NCAM and DAT (dopamine transporter). (See, e.g., US 2017/0014450, US 2017/0102397, U.S. Pat. No. 9,958,460). neuronally derived exosomes can be isolated using affinity capture methods. Such methods include, for example, paramagnetic beads attached to antibodies against specific markers such as L1 CAM. (See, e.g., Shi et al., “Plasma exosomal α-alpha-synuclein is likely CNS derived and increased in Parkinson's disease”, Acta Neuropathol. 2014 November; 128(5): 639-650.)

### D. Exosome Contents

Many proteins, including kinases, linked to the pathogenesis of human neurodegenerative disease, are generated outside the CNS as well as within the brain, and can become attached to the external surface of exosomes that pass through the blood brain barrier into the peripheral circulation. Accordingly, in certain embodiments of the methods disclosed herein, an exosomal fraction is treated to remove molecules bound to the exosomal surface. This can be done, for example, by stringent washing procedures, such as with a Phosphate Buffer Solution (PBS). After such processing, the contents of the exosome can be processed for the assay.

The scrubbed exosomes can then be lysed, and their internal contents released for analysis.

### IV. Determining Diagnosis, Stage, Progression, Prognosis and Risk of Developing of Neurodegenerative Conditions

Biomarker profiles comprising amounts of biomarkers in a biological sample selected from (1) at least one signaling kinase and, optionally, at least one oligomeric form of a neurodegenerative protein, or (2) each of one or a plurality of different signaling kinases, and a change in the profiles over time, indicate presence, severity and direction of neurogenerative conditions of the neurodegenerative type. In particular, abnormal ratios, e.g., elevated amounts, of the protein biomarker disclosed herein indicate a process of neurodegeneration. This process, unchecked, can lead to manifest symptoms in syncucleinopathic conditions. Accordingly, provided herein are methods of ascertaining in a subject (e.g., in either symptomatic or asymptomatic individuals) a diagnosis, stage, progression, rate, prognosis, drug responsiveness and risk of developing a neurodegenerative condition characterized by the abnormal amounts of one or more biomarker proteins (each, referred to herein as a “neuropathic state”, e.g. “syncucleinopathic state”, “amyloidopathic state”, “tauopathic state”, “Huntington's state”).

As used herein, the term “diagnosis” refers to a classification of an individual as having or not having a particular pathogenic condition, including, e.g., the stage of that condition.

As used herein, the term “clinically similar but etiologically different” refers to conditions that share clinical signs and/or symptoms, but which arise from different biological causes.

As used herein, the term “stage” refers to the relative degree of severity of a condition, for example, suspected disease, an early stage, a middle stage or an advanced stage. Staging can be used to group patients based on etiology, pathophysiology, severity, etc.

As used herein, the term “progression” refers to a change, or lack thereof, in stage or severity of a condition over time. This includes an increase, a decrease or stasis in severity of the condition. In certain embodiments, rates of progression, that is, change over time, are measured.

As used herein, the term “prognosis” refers to the predicted course, e.g., the likelihood of progression, of the condition. For example, a prognosis may include a prediction that severity of the condition is likely to increase, decrease or remain the same at some future point in time. In the context of the present disclosure, prognosis can refer to the likelihood that an individual: (1) will develop a neurodegenerative condition, (2) will progress from one stage to another, more advanced, stage of the condition, (3) will exhibit a decrease in severity of the condition, (4) will exhibit functional decline at a certain rate, (5) will survive with a condition for a certain period of time (e.g., survival rate) or (6) will have recurrence of the condition. The condition can be a syncucleinopathic condition (e.g., PD, Lewy body dementia, multiple system atrophy or some related synucleinopathy), an amyloidopathic condition (e.g., Alzheimer's disease), a tauopathic condition (e.g., Alzheimer's disease), and Huntington's disease. These terms are not intended to be absolute, as will be appreciated by any one of skill in the field of medical diagnostics.

As used herein, the term “risk of developing” refers to a probability that an individual who is asymptomatic or preclinical will develop to a definitive diagnosis of disease. Determining probability includes both precise and relative probabilities such as “more likely than not”, “highly likely”, “unlikely”, or a percent chance, e.g., “90%”. Risk can be compared with the general population or with a population matched with the subject based on any of age, sex, genetic risk, and environmental risk factors. In such a case, a subject can be determined to be at increased or decreased risk compared with other members of the population. A subject at increased risk of developing a neurodegenerative condition is likely to positively respond to treatment for a neurodegenerative condition, for example, by prevention of developing the condition, delayed onset of the condition or reduced severity of symptoms or morbidity associated with the condition.

### V. Modeling Profiles of Kinases to Infer Diagnosis, Stage, Progression, Prognosis and Risk of Developing of Neurodegenerative Conditions

Determining diagnosis, stage, progression rate, prognosis and risk of a neurodegenerative condition are processes of classifying a subject into different conditions or different classes or conditions within a state, such as disease/health (diagnosis), stage I/stage II/stage III (stage), likely to remiss/likely to progress (prognosis) or assigning a score on a range. Methods of classification using biomarker profiles can involve identifying profiles that are characteristic of various states and correlating a profile from a subject with class or state. Identifying such profiles can involve analysis of biomarker profiles from subjects belonging to different states and discerning patterns or differences between the profiles. Analysis can be done by visual examination of the profiles or by statistical analysis.

### A. Statistical Analysis

Typically, analysis involves statistical analysis of a sufficiently large number of samples to provide statistically meaningful results. Any statistical method known in the art can be used for this purpose. Such methods, or tools, include, without limitation, correlational, Pearson correlation, Spearman correlation, chi-square, comparison of means (e.g., paired T-test, independent T-test, ANOVA) regression analysis (e.g., simple regression, multiple regression, linear regression, non-linear regression, logistic regression, polynomial regression, stepwise regression, ridge regression, lasso regression, elasticnet regression) or non-parametric analysis (e.g., Wilcoxon rank-sum test, Wilcoxon sign-rank test, sign test). Such tools are included in commercially available statistical packages such as MATLAB, JMP Statistical Software and SAS. Such methods produce models or classifiers which one can use to classify a particular biomarker profile into a particular state.

Statistical analysis can be operator implemented or implemented by machine learning.

### B. Machine Learning

In certain embodiments statistical analysis is enhanced through the use of machine learning tools. Such tools employ learning algorithms, in which the relevant variable or variables are measured in the different possible states, and patterns differentiating the states are determined and used to classify a test subject. Accordingly, any classification method of this disclosure can be developed by comparing measurements of one or more variables in subjects belonging to the various conditions within a particular syncucleinopathic state. This includes, for example, determining a biomarker profile comprising amounts of biomarkers selected from selected from (1) at least one signaling kinase and, optionally, at least one oligomeric form of a neurodegenerative protein, or (2) each of one or a plurality of different signaling kinases in subjects with various diagnoses or at various stages at various times to allow prediction of diagnosis, stage, progression, prognosis, drug responsiveness or risk. Other variables can be included as well, such as family history, lifestyle, exposure to chemicals, various phenotypic traits, etc.

### 1. Training Dataset

A training dataset is a dataset typically comprising a vector of measures for each of a plurality of features for each of a plurality of subjects (more generally referred to as objects). One of the features can be a classification of the subject, for example, a diagnosis or a measure of a degree on a scale. This can be used in supervised learning methods. Other features can be, for example, measured amounts of biomarkers selected from (1) at least one signaling kinase and, optionally, at least one oligomeric form of a neurodegenerative protein, or (2) each of one or a plurality of different signaling kinases. So, for example, a vector for an individual subject can include a diagnosis of a neurodegenerative condition (e.g., diagnosed as having or not having Parkinson's Disease) and measurements of each of a plurality of biomarkers as described herein. In certain embodiments, the training dataset used to generate the classifier comprises data from at least 100, at least 200, or at least 400 different subjects. The ratio of subjects classified has having versus not having the condition can be at least 2:1, at least 1:1, or at least 1:2. Alternatively, subjects pre-classified as having the condition can comprise no more than 66%, no more than 50%, no more than 33% or no more than 20% of subjects.

### 2. Learning Algorithms

Learning algorithms, also referred to as machine learning algorithms, are computer-executed algorithms that automate analytical model building, e.g., for clustering, classification or profile recognition. Learning algorithms perform analyses on training datasets provided to the algorithm.

Learning algorithms output a model, also referred to as a classifier, classification algorithm or diagnostic algorithm. Models receive, as input, test data and produce, as output, an inference or a classification of the input data as belonging to one or another class, cluster group or position on a scale, such as diagnosis, stage, prognosis, disease progression, responsiveness to a drug, etc.

A variety of machine learning algorithms can be used to infer a condition or state of a subject. Machine learning algorithms may be supervised or unsupervised. Learning algorithms include, for example, artificial neural networks (e.g., back propagation networks), discriminant analyses (e.g., Bayesian classifier or Fischer analysis), support vector machines, decision trees (e.g., recursive partitioning processes such as CART—classification and regression trees), random forests, linear classifiers (e.g., multiple linear regression (MLR), partial least squares (PLS) regression and principal components regression (PCR)), hierarchical clustering and cluster analysis. The learning algorithm will generate a model or classifier that can be used to make an inference, e.g., an inference about a disease state of a subject.

### 3. Validation

A model may be subsequently validation using a validation dataset. Validation datasets typically include data on the same features as the training dataset. The model is executed on the training dataset and the number of true positives, true negatives, false positives and false negatives is determined, as a measure of performance of the model.

The model can then be tested on a validation dataset to determine its usefulness. Typically, a learning algorithm will generate a plurality of models. In certain embodiments, models can be validated based on fidelity to standard clinical measures used to diagnose the condition under consideration. One or more of these can be selected based on its performance characteristics.

### C. Computers

Classification of a subject's condition based on any of the states described herein can be performed by a programmable digital computer. The computer can include tangible memory that receives and, optionally stores at least measurements of one or a plurality of oligomeric forms and, optionally, monomeric forms of the protein biomarker (e.g., signaling kinases and, optionally, neurodegenerative proteins), in a subject and a processor that processes this data by the execution of code embodying a classification algorithm. The classification algorithm can be the result of operator-implemented or machine learning-implemented statistical analysis.

A system comprises a first computer as described in communication with a communications network configured to transmit data to the computer and/or transmit results of a test, such as a classification as described herein to a remote computer. The communications network can utilize, for example, a high-speed transmission network including, without limitation, Digital Subscriber Line (DSL), Cable Modem, Fiber, Wireless, Satellite and, Broadband over Powerlines (BPL). The system can further comprise a remote computer connected through the communications network to the first computer.

### D. Model Execution and Making an Inference

The model selected can either result from operator executed statistical analysis or machine learning. In any case, the model can be used to make inferences (e.g., predictions) about a test subject. A biomarker profile, for example in the form of a test dataset, e.g., comprising a vector, containing values of features used by the model, can be generated from a sample taken from the test subject. The test dataset can include all of the same features used in the training dataset, or a subset of these features. The model is then applied to or executed on the test dataset. Correlating a biomarker profile with a condition, disease state, a prognosis, a risk of progression, a likelihood of drug response, etc. is a form of executing a model. Correlating can be performed by a person or by a machine. The choice may depend on the complexity of the operation of correlating. This produces an inference, e.g., a classification of a subject as belonging to a class or a cluster group (such as a diagnosis), or a place on a scale (such as likelihood of responding to a therapeutic intervention).

In certain embodiments the classifier will include a plurality of oligomeric protein forms and, typically, but not necessarily, one or more monomeric forms of the neurodegenerative protein. The classifier may or may not be a linear model, e.g., of the form AX+BY+CZ=N, wherein A, B and C are measured amounts of forms X, Y and Z. The classifier may require, for example, support vector machine analysis. For example, the inference model may perform a pattern recognition in which a biomarker profile lies on a scale between normal and abnormal, with various profiles tending more toward normal or toward abnormal. Thus, the classifier may indicate a confidence level that the profile is normal or abnormal. An abnormal biomarker profile can be a profile that, when analyzed by a a classification algorithm, classifies a subject into a non-normal category, such as disease being present, or at increased risk of disease. A measure of a biomarker may be abnormal if the measure lies outside a range considered normal, for example, a deviation from a normal range that is statistically significant.

The classifier or model may generate, from the one or are plurality of forms measured, a single diagnostic number which functions as the model. Classifying a neuropathological state, e.g., syncucleinopathic state (e.g., diagnosis, stage, progression, prognosis and risk) can involve determining whether the diagnostic number is above or below a threshold (“diagnostic level”). For example, the diagnostic number can be a relative amount of two different signaling kinases. That threshold can be determined, for example, based on a certain deviation of the diagnostic number above normal individuals who are free of any sign of a neurodegenerative, e.g., syncucleinopathic, condition. A measure of central tendency, such as mean, median or mode, of diagnostic numbers can be determined in a statistically significant number of normal and abnormal individuals. A cutoff above normal amounts can be selected as a diagnostic level of a neurodegenerative, e.g., synucleinopathic, condition. That number can be, for example, a certain degree of deviation from the measure of central tendency, such as variance or standard deviation. In one embodiment the measure of deviation is a Z score or number of standard deviations from the normal average.

The model can be selected to provide a desired level of sensitivity, specificity or positive predictive power. For example, the diagnostic level can provide a sensitivity of at least any of 80%, 90%, 95% or 98% and/or a specificity of at least any of 80%, 90%, 95% or 98%, and/or a positive predictive value of at least any of 80%, 90%, 95% or 98%. The sensitivity of a test is the percentage of actual positives that test positive. The specificity of a test is the percentage of actual negatives that test negative. The positive predictive value of a test is the probability that a subject that tests positive is an actual positive.

### VI. Development of Therapeutic Interventions to Treat Neurodegenerative Conditions

In another aspect, provided herein are methods to enable the practical development of therapeutic interventions for neurodegenerative conditions, e.g., syncucleinopathic conditions, amyloidopathic conditions, tauopathic conditions, and Huntington's disease. The methods involve, among other things, selecting subjects for clinical trials and determining effectiveness of the therapeutic intervention in a set of subjects.

Methods comprising monitoring the biomarker profiles of neurodegenerative proteins are useful to determine whether an experimental therapeutic intervention is effective in preventing clinical onset or inhibiting subsequent progression of a synucleinopathy, or whether a subject should be entered into a clinical trial to test the efficacy of a drug candidate to treat such conditions. Biomarker profiles or changes in biomarker profiles of the neurodegenerative protein enable the direct determination of treatment effects on the condition, including, e.g., basic disease process.

### A. Subject Enrollment

Clinical trials involve enrollment of subjects for testing the efficacy and safety of a potential therapeutic intervention, such as a pharmaceutical. Typically, subjects are selected to have different conditions of a state, e.g., subjects with or without a diagnosis of disease or at different stages of disease or different subtypes of disease or different prognosis. Clinical trial subjects can be stratified into different groups to be treated the same or differently. Stratification can be based on any number of factors, including, stage of disease. Disease Staging is a classification system that uses diagnostic findings to produce clusters of patients based on such factors as etiology, pathophysiology and severity. It can serve as the basis for clustering clinically homogeneous patients to assess quality of care, analysis of clinical outcomes, utilization of resources, and the efficacy of alternative treatments.

In one method, potential clinical trial subjects are stratified at least in part on biomarker profiles. Thus, for example, subjects having different biomarker profiles (e.g., higher and lower relative amounts) can be assigned to different groups.

The population of subjects in a clinical trial should be sufficient to show whether the drug produces a statistically significant difference in outcome. Depending on this power level, the number of individuals in the trial can be at least 20, at least 100 or at least 500 subjects. Among these, there must be a significant number of individuals exhibiting a biomarker profile consistent with having the neurodegenerative condition (e.g., an increased level of the biomarker. For example, at least 20%, at least 35%, at least 50%, or at least 66% of the subjects may initially have such a biomarker profile (comprising, e.g., various species of signaling kinases). Also, a significant number of subjects are to be divided between class states. For example, at least 20%, at least 35%, at least 50%, at least 66% or 100% of the subjects may initially have a diagnosis of a neurodegenerative condition (e.g., syncucleinopathic condition (e.g., PD), amyloidopathic condition, tauopathic condition and Huntington's disease).

### B. Drug Development

Upon commencement of the clinical trial effectiveness of the therapeutic intervention on the different stratification groups can be rapidly determined as a function of the effect on the biomarker profile that includes biomarkers selected from (1) at least one signaling kinase and, optionally, at least one oligomeric form of a neurodegenerative protein, or (2) each of one or a plurality of different signaling kinases. More specifically, a change in the biomarker profile predicts the clinical effectiveness of the therapeutic intervention. Methods generally involve first testing individuals to determine biomarker profile comprising signaling kinases, and, optionally, neurodegenerative proteins. After the measurements, the therapeutic intervention, e.g., an experimental drug, is administered to at least a subset of the subjects. Typically, at least a subset of the subjects is given a placebo or no treatment. In some cases, subject serve as their own controls, first receiving a placebo, and then, the experimental intervention, or the reverse, for comparison. In certain instances, this can be done in conjunction with administering already recognized forms of treatment. The population can be divided in terms of dosing, timing and rate of administration of the therapeutic intervention. Ethical considerations may require stopping a study when a statistically significant improvement is seen in test subjects. As used herein, “experimental drug” and “drug candidate” refer to an agent having or being tested for a therapeutic effect. A “putative neuroprotective agent” refers to an agent having or being tested to have neuroprotective action.

After administration of the therapeutic intervention, the biomarker profile is determined again.

The therapeutic intervention can be administration of a drug candidate. Using standard statistical methods, it can be determined whether the therapeutic intervention has had a meaningful impact on the biomarker profile comprising biomarkers selected from (1) at least one signaling kinase and, optionally, at least one oligomeric form of a neurodegenerative protein, or (2) each of one or a plurality of different signaling kinases. In general, a statistically significant change, especially a shift toward a normal profile, compared with the initial biomarker profile indicates that the therapeutic intervention is neuroprotective and thus will delay clinical onset, or slow or preferably reverse progression of the neurodegenerative condition (e.g., syncucleinopathic condition, amyloidopathic condition, tauopathic condition, Huntington's disease.

Accordingly, subjects for whom a biomarker profile comprising biomarkers selected from (1) at least one signaling kinase and, optionally, at least one oligomeric form of a neurodegenerative protein, or (2) each of one or a plurality of different signaling kinases can be measured include, for example: (1) Subjects who are asymptomatic for a neurodegenerative condition (e.g., syncucleinopathic condition, amyloidopathic condition, tauopathic condition, Huntington's disease); (2) subjects having minimal neurodegenerative disease symptoms and no signs suggestive of a neurodegenerative condition (e.g., who may be diagnosed with “suspected” or “preclinical” for a neurodegenerative condition, especially when certain genetic and/or environmental risk factors have been identified); (3) subjects having the diagnosis of “probable” neurodegenerative condition and subjects diagnosed (“definitive diagnosis”) with a neurodegenerative condition. These include, for example, (1) subjects who are asymptomatic for a syncucleinopathic condition, (2) subjects having minimal parkinsonian symptoms and no signs suggestive of a synucleinopathic condition (e.g., who may be diagnosed with “suspected” or “preclinical” for PD or some related synucleinopathy, especially when certain genetic and/or environmental risk factors have been identified); (3) subjects having the diagnosis of “probable” synucleinopathy (e.g., PD) and subjects diagnosed (“definitive diagnosis”) with a synucleinopathic condition.

Subjects are typically human but also include nonhuman animals, for example, those used as models for PD, such as, rodents (e.g., mice and rats), cats, dogs, other domesticated quadrupeds (such as horses, sheep and swine), and nonhuman primates (e.g., monkeys). Animal models include both genetic models and models based on the administration of neurotoxins. Neurotoxins used in such models include, for example, 6-hydroxydopamine (6-OHDA) and 1-methyl-1,2,3,6-tetrahydropyridine (MPTP) administration, and paraquat and rotenone. Genetic models include genetic mutations in SNCA (α-syn, PARK1, and 4), PRKN (parkin RBR E3 ubiquitin protein ligase, PARK2), PINK1 (PTEN-induced putative kinase 1, PARK6), DJ-1 (PARK7), and LRRK2 (leucine-rich repeat kinase 2, PARKS).

Clinical trials for neuroprotective therapies for neurodegenerative conditions, such as synucleinopathies require measures that promptly indicate the effectiveness of the potential therapy. Otherwise, the determination of drug efficacy based on clinical observations ordinarily takes many months. Biomarker profiles comprising neurodegenerative protein oligomers and, optionally monomers provide such measures, thus enabling the practical evaluation of disease modifying drug efficacy in subjects suffering from fatal brain disorders such as PD.

### C. Validation

Subjects are said to respond to therapy when they show a clinically significant change in clinical symptoms. Efficacy of a drug being tested is typically validated by clinical measurements, for example, by determining disease symptoms, signs and stages. Such clinical measures include those described herein, such as the modified Hoehn and Yahr staging scale and the Unified Parkinson's Disease Rating Scale (UPDRS). Biomarker profiles as described herein also provide an indication of response to therapy and can do so at much earlier time periods than other forms of clinical evaluation. This will typically happen after the drug has been validated using traditional methods. However, biomarker profiles can be used in addition to or instead of clinical markers to determine efficacy of a drug in a subject or a population of subjects. For example, responses that can be detected by traditional means only about 18 months after initiation of therapy can be detected in biomarker profiles at as little as 12 months, six months or three months after initiation of therapy. Accordingly, in some embodiments determination of response to therapy involves determining a first biomarker profile of the subject at a first time point, administering a therapeutic intervention to the subject; determining a second biomarker profile after administration of the therapeutic intervention e.g., within about any of one month, three months, six months, nine months, 12 months, 15 months, or 18 months of initiation of therapy; and comparing the first and second biomarker profiles to identify changes. No statistically significant difference in the biomarker profiles indicates no response to therapy. A statistically significant change toward a normal biomarker profile indicates a positive response to therapy while a statistically significant change away from a normal profile indicates a negative response to therapy or, progression of the disease. Where a normal profile is known before the therapeutic intervention is initiated, measurement of the first biomarker profile can be dispensed with and the determination can rely on the second biomarker profile.

### VII. Methods of Treatment

Depending on the stage or class of neurodegenerative condition (e.g., syncucleinopathic condition, amyloidopathic condition, tauopathic condition, Huntington's disease) into which a subject is classified based on the biomarker profile as described herein, a subject may be in need of a therapeutic intervention. Provided herein are methods of treating a subject determined, by the methods disclosed herein, to exhibit a neurodegenerative condition (e.g., a syncucleinopathic condition, and amyloidopathic condition, a tauopathic condition, Huntington's disease) with a therapeutic intervention effective to treat the condition. Therapeutic interventions that change and especially those that return levels of signaling kinases and, optionally, neurodegenerative proteins, reflect an effective treatment, e.g., a therapeutic intervention developed by the methods herein, and clinically validated.

As used herein, the terms “therapeutic intervention”, “therapy” and “treatment” refer to an intervention that produces a therapeutic effect, (e.g., is “therapeutically effective”). Therapeutically effective interventions prevent, slow the progression of, delay the onset of symptoms of, improve the condition of (e.g., causes remission of), improve symptoms of, or cure a disease, such as a synucleinopathic condition. A therapeutic intervention can include, for example, administration of a treatment, administration of a pharmaceutical, or a biologic or nutraceutical substance with therapeutic intent. The response to a therapeutic intervention can be complete or partial. In some aspects, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques. Recognizing that certain sub-groups of subjects may not respond to a therapy, one measure of therapeutic effectiveness can be effectiveness for at least 90% of subjects undergoing the intervention over at least 100 subjects.

As used herein, the term “effective” as modifying a therapeutic intervention (“effective treatment” or “treatment effective to”) or amount of a pharmaceutical drug (“effective amount”), refers to that treatment or amount to ameliorate a disorder, as described above. For example, for the given parameter, a therapeutically effective amount will show an increase or decrease in the parameter of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control. Currently, clinically efficacy against the severity of motor symptoms in a parkinsonian subject can be measured using such standardized scales as the UPDRS and the Hoehn and Yahr scale; for metal and cognitive symptoms the ADAS-cog or the MMPI scales. (It is recognized that the utility of such scales does not necessarily depend on the type or nature of the underlying disease condition.)

Thus, according to some methods a subject is first tested for the biomarker profile comprising forms of oligomeric and/or monomeric forms of neurodegenerative proteins in a biological sample from the subject. A classification into an appropriate condition or class is determined based on the biomarker profile. Based on the classification a decision can be made regarding the type, amount, route and timing of administering an optimally effective therapeutic intervention to the subject.

### A. Synucleinopathic Condition

In certain embodiments, a symptom modifying therapeutic intervention for PD (i.e., a symptomatic or palliative treatment) comprises administration of a drug selected from a dopamine agonist (e.g., pramipexole (e.g., Mirapex™), ropinirole (e.g., Requip), rotigotine (e.g., Neupro), apomorphine (e.g., Apokyn)), levodopa, carbidopa-levodopa (e.g., Rytary, Sinemet), a MAO-B inhibitor (e.g., selegiline (e.g., Eldepryl, Zelapar) or rasagiline (e.g., Azilect)), a catechol-O-methyltransferase (COMT) inhibitor (e.g., entacapone (Comtan) or tolcapone (Tasmar)), an anticholinergic (e.g., benztropine (e.g., Cogentin) or trihexyphenidyl), amantadine or a cholinesterase inhibitor (e.g., rivastigmine (Exelon)) or some similar agent or group of agents.

In another embodiment, the drug is a combination a NK1-antagonist and 6-propylamino-4,5,6,7-tetrahydro-1,3-benzothiazole-2-amine. For example, the NK1-antagonist can be rolapitant or aprepitant and the 6-propylamino-4,5,6,7-tetrahydro-1,3-benzothiazole-2-amine is pramipexole dihydrochloride monohydrate. For example, the daily dose of aprepitant can be between 10 mg to 250 mg, and the daily dose of pramipexole dihydrochloride monohydrate can be between from 1.5 mg to 45 mg. (See, e.g., U.S. patent application 2020/0147097. In another embodiment, the drug is combination product comprising delivery of a 5HT3-antagonist in combination with a therapeutically effective daily dose of a 6-propylamino-4,5,6,7-tetrahydro-1,3-benzothiazole-2-amine, e.g., a combination of ondansetron hydrochloride dihydrate and pramipexole dihydrochloride monohydrate. The daily dose of ondansetron hydrochloride dihydrate can be 4 mg to 32 mg and the daily dose of pramipexole can be 1.5 mg to 42 mg. (See, e.g., U.S. Pat. No. 10,799,484.) In certain embodiments, a neuroprotective or disease modifying therapeutic intervention for PD comprises administration of a putatively disease modifying drug as described in any of the following provisional patent applications, incorporated herein by reference in their entirety: Ser. No. 62/477,187, filed Mar. 27, 2017; Ser. No. 62/483,555, filed Apr. 10, 2017; Ser. No. 62/485,082, filed Apr. 13, 2017; Ser. No. 62/511,424, filed May 26, 2017; Ser. No. 62/528,228, filed Jul. 3, 2017; Ser. No. 62/489,016, filed Apr. 24, 2017; Ser. No. 62/527,215, filed Jun. 30, 2017.

### B. Amyloidopathic Condition

In certain embodiments, a symptom modifying therapeutic intervention for an amyloidopathic condition (i.e., a symptomatic or palliative treatment) comprises administration of a drug such as Razadyne® (galantamine), Exelon® (rivastigmine), and Aricept® (donepezil).

### C. Tauopathic Condition

In certain embodiments, a symptom modifying therapeutic intervention for a tauopathic condition (i.e., a symptomatic or palliative treatment) comprises administration of a drug such as Razadyne® (galantamine), Exelon® (rivastigmine), and Aricept® (donepezil) or those cited herein used for the symptomatic treatment of PD.

### D. Huntington's Disease

In certain embodiments, a symptom modifying therapeutic intervention for Huntington's disease (i.e., a symptomatic or palliative treatment) comprises administration of a drug such as tetrabenazine (Austedo® (deutetrabenazine), IONIS-HTT_{Rx}, as well as various neuroleptics and benzodiazepines.

### VIII. Methods of Evaluating Responsiveness to Therapeutic Interventions

In a subject suffering from a neurodegenerative disorder (e.g., a syncucleinopathic condition, an amyloidopathic condition, a tauopathic condition, Huntington's disease) the effectiveness of a therapeutic intervention or the responsiveness of the subject to the therapeutic intervention can be determined by assessing the effect of the therapeutic intervention on the biomarker profile. This includes effectiveness in any neurodegenerative state, e.g., diagnosis, stage, progression, prognosis and risk. A change in the biomarker profile toward a more normal profile indicates effectiveness of the therapeutic intervention.

Use of biomarker profiles comprising a set of biomarkers selected from (1) at least one signaling kinase and, optionally, at least one oligomeric form of a neurodegenerative protein, or (2) each of one or a plurality of different signaling kinases, confers advantages over conventional means (e.g., changes in symptomatology, functional scales or radiologic scans) for judging treatment efficacy in such situations. Not only are such conventional means of judging efficacy insensitive, inexact and semi-quantitative, but typically require long periods (e.g., years) before becoming of sufficient magnitude to accurately measure. Accordingly, the number of potentially useful treatments tested is significantly reduced, and the expense of clinical trials and thus the eventual cost of useful medications is substantially increased

In certain embodiments, the biomarker profile of the protein biomarker species are measured a plurality of times, typically, before, during and after administration of the therapeutic intervention or at a plurality of time points after the therapeutic intervention.

### IX. Kits

In another aspect, provided herein are kits for detecting biomarkers selected from (1) at least one signaling kinase and, optionally, at least one oligomeric form of a neurodegenerative protein, or (2) each of one or a plurality of different signaling kinases, and interpreting the results so obtained. The kits can comprise containers to hold reagents for isolating exosomes from a bodily fluid, reagents for preferentially isolated neuronally derived exosomes from all exosomes, and reagents sufficient to detect the kinases and/or forms of neurodegenerative proteins.

For example, kits for use in detecting and staging a synucleinopathic disease state in a biological sample can comprise reagents, buffers, enzymes, antibodies and other compositions specific for this purpose. Kits can also typically include instructions for use as well as and software for data analysis and interpretation. The kit may further comprise samples that serve as normative standards. Each solution or composition may be contained in a vial or bottle and all vials held in close confinement in a box for commercial sale.

### EXAMPLES

The following examples are offered by way of illustration and not by way of limitation.

### I. Example 1: Kinases are Differently Active in Syncucleinopathic Conditions

A cohort of individuals who have been diagnosed with a syncucleinopathic condition and are given an active therapeutic intervention and then one that is different, possibly known to be inactive, or the reverse, are the subject of study. Or, a cohort comprising a plurality of subjects who are asymptomatic for a syncucleinopathic condition in a plurality of subjects who have been diagnosed with the syncucleinopathic condition are the subject of study. In either case, venous blood samples are is taken from each subject by venipuncture at various times, including under baseline or control (e.g., inactive intervention treatment) conditions and again during the administration of a potentially active (e.g., experimental intervention) treatment. neuronally derived exosomes are isolated from the blood using methods described herein. Amounts of biomarkers selected from (1) at least one signaling kinase and, optionally, at least one oligomeric form of a neurodegenerative protein, or (2) each of one or a plurality of different signaling kinases are measured that are contained within the isolated exosomes. A pattern of expression is determined. Results show that in the cohort of subjects diagnosed with the syncucleinopathic condition the activity of the signaling kinases and oligomeric forms of neurodegenerative proteins are different to a statistically significant degree. Those found to have a significant change in the results of this biomarker assay are later found to have a proportional change in clinical state.

### II. Example 2: Subject Stratification/Clinical Trial

Volunteer subjects without PD and with PD are tested to determine amounts of biomarkers selected from (1) at least one signaling kinase and, optionally, at least one oligomeric form of a neurodegenerative protein, or (2) each of one or a plurality of different signaling kinases in neuronally derived exosomes. Based on the amounts determined, and using cutoffs determined in the example above, the subjects are clustered into several test groups. Certain test groups are given a placebo. Other test groups are administered different amounts of a compound in a clinical trial. During and/or after administration, the tests are repeated. Collected measurements are analyzed. It is determined that the therapeutic intervention produces a statistically significant change in the activities of the signaling kinases and amounts of oligomeric forms of neurodegenerative proteins.

### Example 3: Clinical Trial for Drug Candidate That Is Neuroprotective for Synucleinopathies

The goal of a Phase II study is to evaluate the safety, tolerability and initial efficacy of pramipexole, given with Aprepitant and with or without and, optionally lovastatin or similarly effective drugs, in patients with PD and related disorders. A sequential treatment, rising-dose, cross-over, out-patient trial in up to 30 patients with PD (PD), Multiple system atrophy (MSA), Lewy body dementia (LBD), or related syncucleinopathic disorder is performed. None of the participants is allowed to have been treated with a dopamine agonist or other centrally active pharmaceutical during the 3 months prior to study entry, except for levodopa-carbidopa (Sinemet), which is maintained at a stable dose throughout the trial to a degree considered medically acceptable. Following baseline clinical and laboratory evaluations, including the United PD Rating Scale (UPDRS-Part Ill) and biomarker protein determinations, consenting individuals meeting accession criteria are switched from their pre-study PD treatment regimen to one that incudes pramipexole ER and Aprepitant. The pramipexole ER dose is titrated to that which is optimally tolerated (or a maximum of 9 mg/day) and then stably maintained for up to about 12 to 16 weeks. Co-treatment with an additional drug (e.g., a statin) given at its maximum approved dose may then begin for an additional 3 months as deemed clinically appropriate, at which time all subjects are returned to their preadmission treatment regimen. During the trial, baseline efficacy and safety measures were repeated at regular intervals including determination of biomarker levels. Efficacy is determined as a function of statistically significant change toward normal of a biomarker profile comprising biomarkers selected from (1) at least one signaling kinase and, optionally, at least one oligomeric form of a neurodegenerative protein, or (2) each of one or a plurality of different signaling kinases.

### IV. Example 4: Diagnosis

A subject presents having certain symptoms consistent with PD but, at a preclinical level when still lacking many of the distinguishing clinical features of this illness. Blood is taken from the subject through venipuncture. Amounts of biomarkers selected from (1) at least one signaling kinase and, optionally, at least one oligomeric form of a neurodegenerative protein, or (2) each of one or a plurality of different signaling kinases are measured from neuronally derived exosomes in the blood. A biomarker profile is determined. A diagnostic algorithm classifies the profile to be consistent with a diagnosis of PD. The subject is diagnosed with PD, and is placed on a therapeutic regimen, either a palliative to mitigate symptoms, or treatment directed to the etiology of the disease for purposes of neuroprotection.

### V. Example 5: Staging

A subject presents with a diagnosis of PD. The doctor orders a blood test on the subject to determine a biomarker profile comprising biomarkers selected from (1) at least one signaling kinase and, optionally, at least one oligomeric form of a neurodegenerative protein, or (2) each of one or a plurality of different signaling kinases. Based on the biomarker profile, the doctor determines that the subject is at an early stage of PD and thus more responsive to a particular therapeutic intervention.

### VI. Example 6: Prognosis/Progression

A subject presents with a diagnosis of PD. The doctor orders first and second blood tests on the subject several months apart to determine a biomarker profile comprising biomarkers selected from (1) at least one signaling kinase and, optionally, at least one oligomeric form of a neurodegenerative protein, or (2) each of one or a plurality of different signaling kinases. Based on the biomarker profile, the doctor determines that the subject's disease is progressing slowly and that the subject is expected to have many years of useful life, even without a risky therapeutic intervention.

### VII. Example 7: Risk Assessment

A subject presents for a physical exam having no symptoms of a synucleinopathic disease. In this case, this individual is aware of a genetic or environmental risk factor. The doctor orders a blood test on the subject to determine a biomarker profile comprising biomarkers selected from (1) at least one signaling kinase and, optionally, at least one oligomeric form of a neurodegenerative protein, or (2) each of one or a plurality of different signaling kinases. Based on the relatively normal biomarker profile of some or all measurable species, compared to healthy control individuals, the doctor determines that the subject has a low probability of developing PD.

### VIII. Example 8: Response to Therapy

A subject presents with a diagnosis of PD. The doctor orders initial blood tests on the subject to determine a biomarker profile comprising biomarkers selected from (1) at least one signaling kinase and, optionally, at least one oligomeric form of a neurodegenerative protein, or (2) each of one or a plurality of different signaling kinases before treatment commences. After a round of treatment, but before clinical symptoms have changed, the doctor orders a second blood test. Based on a change towards normal in the profile, the doctor determines that the treatment is effective or whether the dose needs to be changed or repeated.

### IX. Example 9: Development of Diagnostic

Volunteer subjects without PD and with PD at different diagnosed stages are tested to determine a biomarker profile comprising biomarkers selected from (1) at least one signaling kinase and, optionally, at least one oligomeric form of a neurodegenerative protein, or (2) each of one or a plurality of different signaling kinases. Based on the biomarker profile determined, subjects are classified as showing presence or absence of disease and, optionally stage of disease. Profiles are determined using a computerized learning algorithm that, after data analysis, generates a classification algorithm that infers a diagnosis. The inference model is selected to produce a test with a desired sensitivity and specificity.

### X. Example 10: Biomarker Profiles Are Changed in Syncucleinopathic Conditions

A cohort of individuals who are the subject of study have been diagnosed with a syncucleinopathic condition. The subjects are given an active therapeutic intervention and then one that is different, possibly known to be inactive. Alternatively, the interventions can be given in the reverse order. Or a cohort comprising a plurality of subjects who are asymptomatic for a syncucleinopathic condition in a plurality of subjects who have been diagnosed with the syncucleinopathic condition are the subject of study. In either case, venous blood samples are is taken from each subject by venipuncture at various times, including under baseline or control (e.g., inactive intervention treatment) conditions and again during the administration of a potentially active (e.g., experimental intervention) treatment. neuronally derived exosomes are isolated from the blood using methods described herein. Amounts of biomarkers selected from (1) at least one signaling kinase and, optionally, at least one oligomeric form of a neurodegenerative protein, or (2) each of one or a plurality of different signaling kinases that are contained within the isolated exosomes are measured. These data are combined into a dataset. The dataset is analyzed using statistical methods, in this case, used to train a learning algorithm, e.g., a support vector machine, to develop a model that infers whether a subject should be classified as having or not having the syncucleinopathic condition. Results show that in the cohort of subjects diagnosed with the syncucleinopathic condition certain species of signaling kinases have different activity to a statistically significant degree relative to other signaling kinases. Also, oligomeric forms of neurodegenerative proteins also are changed to a statistically significant degree. Those found to have a significant change in the results of this biomarker assay are later found to have a proportional change in clinical state.

### XI. Example 11: Subject Stratification/Clinical Trial

Volunteer subjects without PD and with PD are tested to determine a biomarker profile comprising biomarkers selected from (1) at least one signaling kinase and, optionally, at least one oligomeric form of a neurodegenerative protein, or (2) each of one or a plurality of different signaling kinases in neuronally derived exosomes. Based on the biomarker profile determined, and using a classifier determined in the example above, the subjects are clustered into several test groups. Certain test groups are given a placebo. Other test groups are administered different amounts of a compound in a clinical trial. During and, optionally after administration, the tests are repeated. Collected measurements are analyzed. It is determined that the therapeutic intervention produces a statistically significant change toward normal of biomarker profiles.

### XII. Example 12: Clinical Trial for Drug Candidate that is Neuroprotective for Synucleinopathies

The goal of a Phase II study is to evaluate the safety, tolerability and initial efficacy of pramipexole, given with Aprepitant and with or without and, optionally lovastatin or similarly effective drugs, in patients with PD and related disorders. A sequential treatment, rising-dose, cross-over, out-patient trial in up to 30 patients with PD (PD), Multiple system atrophy (MSA), Lewy body dementia (LBD), or related syncucleinopathic disorder is performed. None of the participants is allowed to have been treated with a dopamine agonist or other centrally active pharmaceutical during the 3 months prior to study entry, except for levodopa-carbidopa (Sinemet), which is maintained at a stable dose throughout the trial to a degree considered medically acceptable. Following baseline clinical and laboratory evaluations, including the United PD Rating Scale (UPDRS-Part III) and biomarker determinations comprising biomarkers selected from (1) at least one signaling kinase and, optionally, at least one oligomeric form of a neurodegenerative protein, or (2) each of one or a plurality of different signaling kinases, consenting individuals meeting accession criteria are switched from their pre-study PD treatment regimen to one that incudes pramipexole ER and Aprepitant. The pramipexole ER dose is titrated to that which is optimally tolerated (or a maximum of 9 mg/day) and then stably maintained for up to about 12 to 16 weeks. Co-treatment with an additional drug (e.g., a statin) given at its maximum approved dose may then begin for an additional 3 months as deemed clinically appropriate, at which time all subjects are returned to their preadmission treatment regimen. During the trial, baseline efficacy and safety measures were repeated at regular intervals including determination of biomarker levels. Efficacy is determined as a function of statistically significant change toward normal of a biomarker profile.

### XIII. Example 13: Diagnosis

A subject presents having certain symptoms consistent with PD but, at a preclinical level when still lacking many of the distinguishing clinical features of this illness. Blood is taken from the subject through venipuncture. Amounts of biomarkers selected from (1) at least one signaling kinase and, optionally, at least one oligomeric form of a neurodegenerative protein, or (2) each of one or a plurality of different signaling kinases are measured from neuronally derived exosomes in the blood. A biomarker profile is determined. A diagnostic algorithm classifies the profile to be consistent with a diagnosis of PD. The subject is diagnosed with PD, and is placed on a therapeutic regimen, either a palliative to mitigate symptoms, or treatment directed to the etiology of the disease for purposes of neuroprotection.

### XIV. Example 14: Staging

A subject presents with a diagnosis of PD. The doctor orders a blood test on the subject to determine a biomarker profile comprising biomarkers selected from (1) at least one signaling kinase and, optionally, at least one oligomeric form of a neurodegenerative protein, or (2) each of one or a plurality of different signaling kinases. Based on the biomarker profile, the doctor determines that the subject is at an early stage of PD and thus more responsive to a particular therapeutic intervention.

### XV. Example 15: Prognosis/Progression

A subject presents with a diagnosis of PD. The doctor orders first and second blood tests on the subject several months apart to determine a biomarker profile comprising biomarkers selected from (1) at least one signaling kinase and, optionally, at least one oligomeric form of a neurodegenerative protein, or (2) each of one or a plurality of different signaling kinases. Based on the biomarker profile, the doctor determines that the subject's disease is progressing slowly and that the subject is expected to have many years of useful life, even without a risky therapeutic intervention.

### XVI. Example 16: Risk Assessment

A subject presents for a physical exam having no symptoms of a synucleinopathic disease. In this case, this individual is aware of a genetic or environmental risk factor. The doctor orders a blood test on the subject to determine a biomarker profile comprising biomarkers selected from (1) at least one signaling kinase and, optionally, at least one oligomeric form of a neurodegenerative protein, or (2) each of one or a plurality of different signaling kinases. Based on the relatively abnormal biomarker profile of some or all measurable species of biomarkers compared to healthy control individuals, the doctor determines that the subject has a low probability of developing PD.

### XVII. Example 17: Response to Therapy

A subject presents with a diagnosis of PD. The doctor orders initial blood tests on the subject to determine a biomarker profile comprising biomarkers selected from (1) at least one signaling kinase and, optionally, at least one oligomeric form of a neurodegenerative protein, or (2) each of one or a plurality of different signaling kinases before treatment commences. After a round of treatment, but before clinical symptoms have changed, the doctor orders a second blood test. Based on a change towards normal in the a, the doctor determines that the treatment is effective or whether the dose needs to be changed or repeated.

As used herein, the following meanings apply unless otherwise specified. The word “may” is used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The term “or” is, unless indicated otherwise, non-exclusive, i.e., encompassing both “and” and “or.” The term “any of” between a modifier and a sequence means that the modifier modifies each member of the sequence. So, for example, the phrase “at least any of 1, 2 or 3” means “at least 1, at least 2 or at least 3”. The phrase “at least one” includes “a plurality”. The term “consisting essentially of” refers to the inclusion of recited elements and other elements that do not materially affect the basic and novel characteristics of a claimed combination.

While certain embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

All publications and patent applications mentioned in this specification are herein incorporated by reference to the same extent as if each individual publication or patent application were specifically and individually indicated to be incorporated by reference.

What is claimed is:

1. A method comprising:
a) enriching each biological sample in a collection of biological samples for neuronally derived microparticles, e.g., exosomes, wherein:
(i) the collection of biological samples is from subjects in a cohort of subjects, wherein the cohort comprises subjects including:
(1) a plurality of subjects diagnosed with a neurodegenerative condition at each of a plurality of different disease stages, wherein each of the diagnosed subjects has received a putative neuroprotective agent, and/or
(2) a plurality of healthy control subjects,
wherein the biological samples were collected before and again at one or more times during and, optionally, after administration of the putative neuroprotective agent;
b) isolating protein contents from an internal compartment of the microparticles, e.g., exosomes, to produce a biomarker sample;
c) measuring, in the biomarker sample, a set of biomarkers to create a dataset, wherein the set of biomarkers includes:
(i) at least one signaling kinase and, optionally, at least one oligomeric form of a neurodegenerative protein; or
(ii) a plurality of different signaling kinases; and
d) performing statistical analysis on the dataset to compare differences in the biomarker sets:
(i) in individual subjects over time to determine a diagnostic algorithm that predicts rates of disease progression or degree of response to the putative neuroprotective agent; or
(ii) between different subjects to determine a diagnostic algorithm that (1) makes a pathogenic diagnosis, (2) separates clinically similar but etiologically different neurodegenerative disorder subgroups, or (3) predicts whether or the degree to which a subject is likely to respond to the putative neuroprotective agent.

2. The method of claim 1, further comprising, before enriching:
I) providing a cohort of subjects, wherein the cohort comprises subjects including: (i) a plurality of subjects diagnosed with a neurodegenerative condition at each of a plurality of different disease stages, and/or (ii) a plurality of healthy control subjects;
II) administering to each of the diagnosed subjects a putative neuroprotective agent;
III) before and again at one or more times during and, optionally, after administration of the putative neuroprotective agent, collecting a biological sample from each of the subjects in the cohort.

3. The method of claim 1, wherein at least one of the signaling kinases is a kinase of the PI3K-Akt-mTOR signaling pathway.

4. The method of claim 1, wherein at least one of the signaling kinases is selected from mitogen-activated protein kinase (MAPK or MEK), extracellular signal-regulated kinases (ERK), glycogen synthase kinase 3 beta (GSK3B), AKT kinase and beclin.

5. The method of claim 1, wherein the neurodegenerative protein selected from alpha synuclein, amyloid beta, tau, or huntingtin.

6. The method of claim 1, wherein the oligomeric form of the neurodegenerative protein is a collection of oligomeric forms, e.g., oligomers of alpha synuclein, e.g., alpha synuclein 2-50, e.g., alpha synuclein 4-30, e.g., alpha synuclein 4-20.

7. The method of claim 1, further comprising:
e) validating one or more of the diagnostic algorithms against standard clinical measures.

8. The method of claim 1, wherein the statistical analysis comprises: correlational, Pearson correlation, Spearman correlation, chi-square, comparison of means (e.g., paired T-test, independent T-test, ANOVA) regression analysis (e.g., simple regression, multiple regression, linear regression, non-linear regression, logistic regression, polynomial regression. stepwise regression, ridge regression, lasso regression, elasticnet regression) or non-parametric analysis (e.g., Wilcoxon rank-sum test, Wilcoxon sign-rank test, sign test).

9. The method of any of claims 1-8, wherein the statistical analysis is executed by computer.

10. The method of claim 9, wherein the statistical analysis comprises machine learning.

11. The method of claim 1, wherein the subjects are humans.

12. The method of claim 1, wherein the neurodegenerative condition is a synucleinopathic disorder.

13. The method of claim 12, wherein the synucleinopathic disorder is Parkinson's disease.

14. The method of claim 12, wherein the synucleinopathic disorder is Lewy body dementia.

15. The method of claim 13, wherein the standard clinical measures are selected from UPDRS scores, CGI scores and radiologic findings.

16. The method of claim 1, wherein the neurodegenerative condition is an amyloidopathy, a tauopathy or Huntington's disease.

17. The method of claim 1, wherein the biological sample comprises a venous blood sample.

18. The method of claim 1, wherein the different disease stages comprise one or more of suspected, early, middle, and clinically advanced.

19. The method of claim 1, wherein the times during or after administration are selected from 1, 2, 3 or more months after treatment.

20. The method of claim 1, wherein enriching comprises using one or more brain-specific protein markers.

21. The method of claim 20, wherein at least one of the brain-specific markers comprises K1 cam.

22. The method of claim 1, wherein isolating comprises washing the exosomes in each enriched sample to remove surface membrane-bound proteins.

23. The method of claim 22, wherein the exosomes are washed with PBS.

24. The method of claim 1, wherein the forms of the neurodegenerative protein are measured by gel electrophoresis, Western blot or fluorescence techniques.

25. A method comprising:
a) enriching a biological sample from a subject for neuronally derived microparticles, e.g., exosomes;
b) isolating protein contents from an internal compartment of the microparticles, e.g., exosomes, to produce a biomarker sample;
c) measuring, in the biomarker sample, a set of biomarkers to create a dataset, wherein the set of biomarkers includes:
(1) at least one signaling kinase and, optionally, at least one oligomeric form of a neurodegenerative protein; or
(2) a plurality of different signaling kinases; and
d) using the dataset to perform one of the following: (1) make a pathogenic diagnosis, (2) classify the subject into one of a plurality of clinically similar but etiologically different neurodegenerative disorder subgroups, or (3) predict whether or the degree to which the subject is likely to respond to the putative neuroprotective agent.

26. The method of claim 25, wherein using comprises executing a diagnostic algorithm of claim 1, on the dataset.

27. The method of claim 25, wherein at least one of the signaling kinases is a kinase of the PI3K-Akt-mTOR signaling pathway.

28. The method of claim 25, wherein at least one of the signaling kinases is selected from mitogen-activated protein kinase (MAPK or MEK), extracellular signal-regulated kinases (ERK), glycogen synthase kinase 3 beta (GSK3B), AKT kinase and beclin.

29. The method of claim 25, wherein the neurodegenerative protein selected from alpha synuclein, amyloid beta, tau, or huntingtin.

30. The method of claim 25, wherein the oligomeric form of the neurodegenerative protein is a collection of oligomeric forms, e.g., oligomers of alpha synuclein, e.g., alpha synuclein 2-50, e.g., alpha synuclein 4-30, e.g., alpha synuclein 4-20.

31. The method of claim 25, wherein isolating neuronally derived exosomes comprises:
(i) ultra-centrifugation;
(ii) density gradient centrifugation; or
(iii) size exclusion chromatography.

32. The method of claim 25, wherein isolating neuronally derived exosomes comprises capturing the neuronally derived exosomes using a binding moiety that binds to brain-specific protein.

33. The method of claim 32, wherein the brain-specific protein is L1CAM.

34. The method of claim 25, wherein removing proteins from the surface of the isolated exosomes comprises washing the isolated exosomes with an aqueous solution (e.g., phosphate buffered saline (“PBS”)).

35. The method of claim 25, wherein determining amounts of a neurodegenerative protein comprises:
i) separating species of oligomeric α-synuclein into a plurality of fractions;
ii) measuring each of one or a plurality of the separated oligomeric α-synuclein species and, optionally, one or a plurality of species selected from: monomeric α-synuclein, tau-synuclein co-polymers, amyloid beta-synuclein co-polymers and tau-amyloid beta-synuclein co-polymers.

36. The method of claim 35, wherein separating species into a plurality of fractions comprises separating by electrophoresis.

37. The method of claim 35, wherein separating species into a plurality of fractions comprises separating by chromatography.

38. The method of claim 35, wherein determining among the separated species, at least one oligomeric form of α-synuclein selected from forms having between 2 and about 100 monomeric units, between 4 and 16 monomeric units and no more than about 30 monomeric units.

39. The method of claim 35, wherein determining among the separated species, a quantitative measure of monomeric α-synuclein.

40. The method of claim 35, wherein measuring among the separated species, a plurality of different oligomeric α-synuclein species.

41. The method of claim 35, wherein measuring among the separated species a co-polymer comprising α-synuclein and tau.

42. The method of claim 35, wherein determining among the separated species, a quantitative measure of a co-polymer comprising α-synuclein and amyloid beta.

43. The method of claim 35, wherein measuring the separated species comprises detecting one or a plurality of separated species by immunoassay.

44. The method of claim 43, wherein the immunoassay comprises immunoblotting.

45. The method of claim 43, wherein the immunoassay comprises Western blot.

46. The method of claim 43, wherein the immunoassay uses an antibody coupled to a direct label.

47. The method of claim 43, wherein the immunoassay uses an antibody coupled to an indirect label.

48. The method of claim 25, further comprising:
I) measuring the biomarkers in the subject before and after administration of a putative neuroprotective agent; and
II) determining changes in amounts of proteins or patterns of biomarkers, wherein changes toward normal amounts or patterns indicate efficacy of the neuroprotective agent.

49. The method of claim 25, further comprising:
measuring the biomarkers in the subject at two different times; and
determining changes in amounts of proteins or patterns of biomarkers, wherein changes indicate a change in a neurodegenerative state.

50. The method of claim 25, comprising collecting a plurality of biological samples from the subject over a time period, optionally wherein the subject is receiving a putative or known neuroprotective agent during the time period, wherein the diagnostic algorithm predicts rates of disease progression or degree of response to the putative neuroprotective agent.

51. A method comprising:
a) providing a dataset comprising, for each of a plurality of subjects, values indicating (1) state of a neurodegenerative condition, and (2) measures of a set of biomarkers, wherein the set of biomarkers includes:
(i) at least one signaling kinase and, optionally, at least one oligomeric form of a neurodegenerative protein; or
(ii) a plurality of different signaling kinases; and
b) performing a statistical analysis on the dataset to develop a model that infers the state of the neurodegenerative condition in an individual.

52. The method of claim 51, wherein at least one of the signaling kinases is a kinase of the PI3K-Akt-mTOR signaling pathway.

53. The method of claim 51, wherein at least one of the signaling kinases is selected from mitogen-activated protein kinase (MAPK or MEK), extracellular signal-regulated kinases (ERK), glycogen synthase kinase 3 beta (GSK3B), AKT kinase and beclin.

54. The method of claim 51, wherein the neurodegenerative protein selected from alpha synuclein, amyloid beta, tau, or huntingtin.

55. The method of claim 51, wherein the oligomeric form of the neurodegenerative protein is a collection of oligomeric forms, e.g., oligomers of alpha synuclein, e.g., alpha synuclein 2-50, e.g., alpha synuclein 4-30, e.g., alpha synuclein 4-20.

56. The method of claim 51, wherein the statistical analysis is performed by computer.

57. The method of claim 51, wherein the statistical analysis is not performed by computer.

58. The method of claim 51, wherein the statistical analysis comprises: correlational, Pearson correlation, Spearman correlation, chi-square, comparison of means (e.g., paired T-test, independent T-test, ANOVA) regression analysis (e.g., simple regression, multiple regression, linear regression, non-linear regression, logistic regression, polynomial regression. stepwise regression, ridge regression, lasso regression, elasticnet regression) or non-parametric analysis (e.g., Wilcoxon rank-sum test, Wilcoxon sign-rank test, sign test).

59. The method of claim 52, wherein the statistical analysis comprises training a machine learning algorithm on the dataset.

60. The method of claim 59, wherein the machine learning algorithm is selected from: artificial neural networks (e.g., back propagation networks), decision trees (e.g., recursive partitioning processes, CART), random forests, discriminant analyses (e.g., Bayesian classifier or Fischer analysis), linear classifiers (e.g., multiple linear regression (MLR), partial least squares (PLS) regression, principal components regression (PCR)), mixed or random-effects models, non-parametric classifiers (e.g., k-nearest neighbors), support vector machines, and ensemble methods (e.g., bagging, boosting).

61. The method of claim 51, wherein the state is selected from diagnosis, stage, prognosis or progression of the neurodegenerative condition.

62. The method of claim 51, wherein the state is measured as a categorical variable (e.g., a binary state or one of a plurality of categorical states).

63. The method of claim 62, wherein the categories comprise a diagnosis consistent with (e.g., positive or diagnosed as having) having the neurodegenerative condition and inconsistent with (e.g., negative or diagnosed as not having) having the neurodegenerative condition.

64. The method of claim 62, wherein the categories comprise different stages of the neurodegenerative condition.

65. The method of claim 51, wherein the state is measured as a continuous variable (e.g., on a scale).

66. The method of claim 61, wherein the continuous variable is a range is or degrees of the neurodegenerative condition.

67. The method of claim 51, wherein the subjects are animals, e.g., fish, avians, amphibians, reptiles, or mammals, e.g., rodents, primates or humans.

68. The method of claim 51, wherein the plurality of subjects is at least any of 10, 25, 50, 100, 200, 400 or 800.

69. The method of claim 51, wherein, for each subject, the sample for which the quantitative measures are determined are taken at a first time point and the state of the neurodegenerative condition is determined at a second, later time point.

70. The method of claim 51, wherein the biological sample comprises blood or a blood fraction (e.g., plasma or serum).

71. The method of claim 51, wherein the neurodegenerative condition is a synucleinopathy, e.g., Parkinson's Disease or Lewy Body Dementia.

72. The method of claim 51, wherein the neurodegenerative condition is an amyloidopathy, e.g., Alzheimer's Disease, a tauopathy, e.g., Alzheimer's Disease or Huntington's disease.

73. A method of inferring a risk of developing, a diagnosis of, a stage of, a prognosis of or a progression of a neurodegenerative condition characterized by a neurodegenerative protein, wherein the method comprises:
a) measuring, from a biological sample from a subject that is enriched for neuronally derived microparticles, e.g., exosomes, a set of biomarkers to create a dataset, wherein the set of biomarkers includes:
(1) at least one signaling kinase and, optionally, at least one oligomeric form of a neurodegenerative protein; or
(2) a plurality of different signaling kinases; and
b) executing a model, e.g., a model of claim 51, on the dataset to infer a risk of developing, a diagnosis of, a stage of, a prognosis of or a progression of the neurodegenerative condition.

74. The method of claim 73, wherein at least one of the signaling kinases is a kinase of the PI3K-Akt-mTOR signaling pathway.

75. The method of claim 73, wherein at least one of the signaling kinases is selected from mitogen-activated protein kinase (MAPK or MEK), extracellular signal-regulated kinases (ERK), glycogen synthase kinase 3 beta (GSK3B), AKT kinase and beclin.

76. The method of claim 73, wherein the neurodegenerative protein selected from alpha synuclein, amyloid beta, tau, or huntingtin.

77. The method of claim 73, wherein the oligomeric form of the neurodegenerative protein is a collection of oligomeric forms, e.g., oligomers of alpha synuclein, e.g., alpha synuclein 2-50, e.g., alpha synuclein 4-30, e.g., alpha synuclein 4-20.

78. The method of claim 73, wherein the neurodegenerative protein forms for which the quantitative measures are determined are selected from:
(I) at least one oligomeric form;
(II) a plurality of oligomeric forms;
(III) at least one oligomeric form and at least one monomeric form;
(IV) a plurality of oligomeric forms and at least one monomeric form;
(V) at least one oligomeric form and a plurality of monomeric forms; and
(VI) a plurality of oligomeric forms and a plurality of monomeric forms.

79. The method of claim 73, wherein at least one of the oligomeric forms comprises a collection of species of the neurodegenerative protein.

80. The method of claim 73, wherein the model comprises comparing relative amounts an oligomeric form to monomeric form of the neurodegenerative protein to relative amounts in a statistically significant number of control individuals.

81. The method of claim 73, wherein the model comprises detecting a pattern of relative amounts of a plurality of the oligomeric forms from which model the inference is made.

82. The method of claim 73, wherein the subject is asymptomatic or preclinical for a neurodegenerative condition.

83. The method of claim 73, wherein the subject presents to a healthcare provider, such as a doctor, during a routine office visit or as part of a doctor's ordinary practice of medicine.

84. The method of claim 73, wherein the model is executed by computer.

85. The method of claim 73, wherein the model is not executed by computer.

86. A method for determining effectiveness of a therapeutic intervention in treating a neurodegenerative condition, wherein the method comprises:
(a) inferring, in each subject in a population comprising a plurality of subjects, an initial state of a neurodegenerative condition by:
(1) measuring, from a biological sample from a subject that is enriched for neuronally derived microparticles, e.g., exosomes, a set of biomarkers to create a dataset, wherein the set of biomarkers includes:
(i) at least one signaling kinase and, optionally, at least one oligomeric form of a neurodegenerative protein; or
(ii) a plurality of different signaling kinases; and
(2) inferring the initial state using a model, e.g., a model of claim 51;
(b) after inferring, administering the therapeutic intervention to the subjects;
(c) after administering, inferring, in each subject individual in the population, a subsequent a subsequent state of the neurodegenerative condition by:
(1) measuring, from a biological sample from a subject that is enriched for neuronally derived microparticles, e.g., exosomes, a set of biomarkers to create a dataset, wherein the set of biomarkers includes;
(i) at least one signaling kinase and, optionally, at least one oligomeric form of a neurodegenerative protein; or
(ii) a plurality of different signaling kinases; and
(2) inferring the subsequent state using the model; and
(d) based on the initial and subsequent inferences in the population, determining that the therapeutic intervention is effective if the subsequent inferences exhibit a statistically significant change toward a normal state compared with the initial inferences, or that the therapeutic intervention is not effective if the subsequent inferences do not exhibit a statistically significant change compared with the initial inferences toward a normal state.

87. The method of claim 86, wherein at least one of the signaling kinases is a kinase of the PI3K-Akt-mTOR signaling pathway.

88. The method of claim 86, wherein at least one of the signaling kinases is selected from mitogen-activated protein kinase (MAPK or MEK), extracellular signal-regulated kinases (ERK), glycogen synthase kinase 3 beta (GSK3B), AKT kinase and beclin.

89. The method of claim 86, wherein the neurodegenerative protein selected from alpha synuclein, amyloid beta, tau, or huntingtin.

90. The method of claim 86, wherein the oligomeric form of the neurodegenerative protein is a collection of oligomeric forms, e.g., oligomers of alpha synuclein, e.g., alpha synuclein 2-50, e.g., alpha synuclein 4-30, e.g., alpha synuclein 4-20.

91. The method of claim 86, wherein the therapeutic intervention comprises administration of a drug or combination of drugs.

92. The method of claim 86, wherein the population comprises at least 20, at least 50, at least 100 or at least 200 subjects, wherein at least 20%, at least 35%, at least 50%, or at least 75% of the subjects initially have elevated relative amounts of oligomeric forms of the protein to monomeric forms of the protein.

93. The method of claim 86, wherein at least 20%, at least 25%, at least 30%, or at least 35%, least 50%, at least 66%, at least 80%, or 100% of the subjects initially have a diagnosis of a neurodegenerative condition.

94. The method of claim 86, wherein the inference is made by computer.

95. The method of claim 86, wherein the inference is not made by computer.

96. A method for qualifying subjects for a clinical trial of a therapeutic intervention for the treatment or prevention of a neurodegenerative condition comprising:
a) determining that a subject is abnormal with respect with a neurodegenerative condition by:
(1) measuring, from a biological sample from a subject that is enriched for neuronally derived microparticles, e.g., exosomes, a set of biomarkers to create a dataset, wherein the set of biomarkers includes;
(i) at least one signaling kinase and, optionally, at least one oligomeric form of a neurodegenerative protein; or
(ii) a plurality of different signaling kinases;
(2) executing a model, e.g., a model of claim 51, on the profile to infer that the subject is abnormal with respect with the neurodegenerative condition; and
b) enrolling the subject in the clinical trial of a potentially therapeutic intervention for said neurodegenerative condition.

97. The method of claim 96, wherein at least one of the signaling kinases is a kinase of the PI3K-Akt-mTOR signaling pathway.

98. The method of claim 96, wherein at least one of the signaling kinases is selected from mitogen-activated protein kinase (MAPK or MEK), extracellular signal-regulated kinases (ERK), glycogen synthase kinase 3 beta (GSK3B), AKT kinase and beclin.

99. The method of claim 96, wherein the neurodegenerative protein is selected from alpha synuclein, amyloid beta, tau, or huntingtin.

100. The method of claim 96, wherein the oligomeric form of the neurodegenerative protein is a collection of oligomeric forms, e.g., oligomers of alpha synuclein, e.g., alpha synuclein 2-50, e.g., alpha synuclein 4-30, e.g., alpha synuclein 4-20.

101. The method of claim 96, wherein the model is executed by computer.

102. The method of claim 96, wherein the model is not executed by computer.

103. A method of monitoring progress of a subject on a therapeutic intervention for a neurodegenerative condition comprising:
(a) inferring, in the subject, an initial state of a neurodegenerative condition by:
(1) determining, from a biological sample from a subject that is enriched for neuronally derived microparticles, e.g., exosomes, measures of a set of biomarkers, wherein the set of biomarkers includes:
(i) at least one signaling kinase and, optionally, at least one oligomeric form of a neurodegenerative protein; or
(ii) a plurality of different signaling kinases; and
(2) executing a model, e.g., a model of claim 51, to infer an initial state of the neurodegenerative condition;
(b) after inferring, administering the therapeutic intervention to the subject;
(c) after administering, inferring, in the subject, a subsequent state of the neurodegenerative condition by:
(1) determining, from a biological sample from a subject that is enriched for neuronally derived microsomal particles, a biomarker profile comprising amounts of each of a plurality of different signaling kinases to create a dataset; and
(2) executing a model, e.g. a model of claim 51, to infer a subsequent state of the neurodegenerative condition;
(d) based on the initial and subsequent state inferences, determining that the subject is responding positively to the therapeutic intervention if the subsequent inference exhibits a change toward a normal state compared with the initial inferences, or that the therapeutic intervention is not effective if the subsequent inferences do not exhibit a change compared with the initial inferences toward a normal state.

104. The method of claim 103, wherein at least one of the signaling kinases is a kinase of the PI3K-Akt-mTOR signaling pathway.

105. The method of claim 103, wherein at least one of the signaling kinases is selected from mitogen-activated protein kinase (MAPK or MEK), extracellular signal-regulated kinases (ERK), glycogen synthase kinase 3 beta (GSK3B), AKT kinase and beclin.

106. The method of claim 103, wherein the neurodegenerative protein selected from alpha synuclein, amyloid beta, tau, or huntingtin.

107. The method of claim 103, wherein the oligomeric form of the neurodegenerative protein is a collection of oligomeric forms, e.g., oligomers of alpha synuclein, e.g., alpha synuclein 2-50, e.g., alpha synuclein 4-30, e.g., alpha synuclein 4-20.

108. The method of claim 103, wherein the model is executed by computer.

109. The method of claim 103, wherein the model is not executed by computer.

110. A method comprising:
(a) determining, by the method of claim 73, that a subject has a neurodegenerative condition, and
(b) administering to the subject a palliative or neuroprotective therapeutic intervention efficacious to treat the condition.

111. The method of claim 110, wherein the therapeutic intervention moves a biomarker profile of the subject toward normal, wherein a movement toward normal indicates neuroprotection.

112. A method comprising administering to a subject determined by the method of claim 73, to have an abnormal pattern of biomarkers, a palliative or neuroprotective therapeutic intervention effective to treat the condition.

113. The method of claim 112, wherein the subject is asymptomatic or preclinical for the neurodegenerative condition.

114. A kit comprising reagents sufficient to detect either:
(1) at least one of signaling kinase and at least one oligomeric form of a neurodegenerative protein; or
(2) a plurality of different signaling kinases.

115. The kit of claim 114, wherein the reagents comprise antibodies.

116. A method of inferring a risk of developing, a diagnosis of, a stage of, a prognosis of or a progression of a neurodegenerative condition, wherein the method comprises:
a) measuring, from a biological sample from a subject that is enriched for neuronally derived microparticles, e.g., exosomes, a set of biomarkers to create a dataset, wherein the set of biomarkers includes:
(1) at least one signaling kinase and, optionally, at least one oligomeric form of a neurodegenerative protein; or
(2) a plurality of different signaling kinases; and
b) correlating the dataset with a risk of developing, a diagnosis of, a stage of, a prognosis of or a progression of the neurodegenerative condition.

117. The method of claim 116, wherein at least one of the signaling kinases is a kinase of the PI3K-Akt-mTOR signaling pathway.

118. The method of claim 116, wherein at least one of the signaling kinases is selected from mitogen-activated protein kinase (MAPK or MEK), extracellular signal-regulated kinases (ERK), glycogen synthase kinase 3 beta (GSK3B), AKT kinase and beclin.

119. The method of claim 116, wherein the neurodegenerative protein selected from alpha synuclein, amyloid beta, tau, or huntingtin.

120. The method of claim 116, wherein the oligomeric form of the neurodegenerative protein is a collection of oligomeric forms, e.g., oligomers of alpha synuclein, e.g., alpha synuclein 2-50, e.g., alpha synuclein 4-30, e.g., alpha synuclein 4-20.

121. A method comprising:
(a) identifying a subject having a neurodegenerative condition or likely to positively respond to a treatment for a neurodegenerative condition, wherein identifying comprises:
(1) measuring, in a sample from the subject enriched for neuronally derived exosomes (e.g., from the internal contents of the exosomes), a set of biomarkers, to create a biomarker profile, wherein the set of biomarkers includes one or a plurality of signaling kinases and, optionally, at least one oligomeric form of a neurodegenerative protein; and
(2) determining, based on an abnormal biomarker profile, that the subject suffers from the neurodegenerative condition; and
(b) administering to the identified subject, an effective amount of a pharmaceutical composition to treat the neurodegenerative condition.

122. The method of claim 121, wherein the neurodegenerative condition is a synucleopathic condition, and the pharmaceutical composition comprises comprising a dopamine agonist (e.g., pramipexole (e.g., Mirapex™), ropinirole (e.g., Requip), rotigotine (e.g., Neupro), apomorphine (e.g., Apokyn)), levodopa, carbidopa-levodopa (e.g., Rytary, Sinemet), a MAO-B inhibitor (e.g., selegiline (e.g., Eldepryl, Zelapar) or rasagiline (e.g., Azilect)), a catechol-O-methyltransferase (COMT) inhibitor (e.g., entacapone (Comtan) or tolcapone (Tasmar)), an anticholinergic (e.g., benztropine (e.g., Cogentin) or trihexyphenidyl), amantadine or a cholinesterase inhibitor (e.g., rivastigmine (Exelon)).

123. The method of claim 121, wherein the synucleopathic condition is Parkinson's Disease.

124. The method of claim 123, wherein the pharmaceutical composition comprises a dopamjne agonist.

125. The method of claim 124, wherein the pharmaceutical composition further comprises an NK1-antagogonist.

126. The method of claim 125, wherein the dopamine agonist is 6-propylamino-4,5,6,7-tetrahydro-1,3-benzothiazole-2-amine and the NK1-antagonist is aprepitant or rolapitant.

127. The method of claim 124, wherein the pharmaceutical composition further comprises an 5HT3-antagonist.

128. The method of claim 127, wherein the dopamine agonist is 6-propylamino-4,5,6,7-tetrahydro-1,3-benzothiazole-2-amine and the 5HT3 antagonist is ondansetron hydrochloride dihydrate.

129. A method comprising administering to a subject characterized as having a biomarker profile indicative of a neurodegenerative condition or being likely to positively respond to a treatment for a neurodegenerative condition, an effective amount of a pharmaceutical composition to treat the neurodegenerative condition; wherein the biomarker panel comprises set of biomarkers includes one or a plurality of signaling kinases and, optionally, at least one oligomeric form of a neurodegenerative protein measured from a sample from the subject enriched for neuronally derived exosomes (e.g., from the internal contents of the exosomes).

130. The method of claim 129, wherein the neurodegenerative condition is Parkinson's Disease, and wherein the pharmaceutical composition comprises a dopamine agonist.
